(19) United States
(12) Patent Application Publication
(10) Pub. No.: US 2021/0315637 A1
(43) Pub. Date: Oct. 14, 2021

(54) ROBOTICALLY-ASSISTED SURGICAL SYSTEM, ROBOTICALLY-ASSISTED SURGICAL METHOD, AND COMPUTER-READABLE MEDIUM

(71) Applicants: Medicaroid Corporation, Kobe-shi (JP); Ziosoft, Inc., Tokyo (JP)

(72) Inventors: Jota IDA, Kobe-shi (JP); Shusuke CHINO, Tokyo (JP); Tsuyoshi NAGATA, Tokyo (JP); Yutaka KARASAWA, Tokyo (JP); Shinichiro SEO, Tokyo (JP)

(21) Appl. No.: 17/225,255

(22) Filed: Apr. 8, 2021

(30) Foreign Application Priority Data
Apr. 9, 2020 (JP) 2020-070567

(51) Int. Cl. A61B 34/10; A61B 34/30

(52) U.S. Cl. CPC A61B 34/10; A61B 2034/107; A61B 34/30

(57) ABSTRACT

A robotically-assisted surgical system that assists robotic surgery by a surgical robot having a robot main body includes one or more processors. The one or more processors are configured to plan a position of a port to be perforated on a body surface of a subject which is a target of the robotic surgery, acquire a captured image obtained by capturing the subject including at least a part of the subject by an overview camera included in the robot main body, recognize a planned position of the port in the captured image based on the captured image and the planned position of the port, and show the captured image and port position information indicating the planned position of the port in the subject illustrated in the captured image, on a display unit.

### CROSS-REFERENCE TO RELATED APPLICATIONS

This application is based on and claims priority under 35 USC 119 from Japanese Patent Application No. 2020-070567 filed on Apr. 9, 2020, the contents of which are incorporated herein by reference.

### TECHNICAL FIELD

The present disclosure relates to a robotically-assisted surgical system, a robotically-assisted surgical method, and a computer-readable medium.

### BACKGROUND ART

In the related art, surgical robots (robotic surgery systems) that perform robotic surgery have been known. For example, the surgical robot includes an attachment base, a plurality of surgical instruments, and a joint support body assembly. Each surgical instrument can be inserted into a patient through the associated minimally invasive aperture to the desired internal surgical site. The joint support body assembly movably supports a plurality of surgical instruments against the attachment base. The joint support body assembly generally includes an orientation platform, a platform linkage that movably supports the orientation platform to the attachment base, and a plurality of manipulators attached to the orientation platform. Each manipulator supports an accompanying instrument in a movable manner (refer to Japanese Unexamined Patent Application Publication No. 2018-196780).

In the surgical robot of Japanese Unexamined Patent Application Publication No. 2018-196780, there is a platform linkage 42 below the orientation platform 36, and the manipulator is attached to the lower portion of the platform linkage. Therefore, in the lower portion of the orientation platform, it is not possible to ensure much workspace because each member of the surgical robot is placed to be congested.

Accordingly, it is difficult to perforate a port for inserting surgical instruments into the body of the patient after the surgical robot is placed in the vicinity of the surgical bed. Therefore, in a small operating room, there is also a case where the surgical robot enters the operating room after the port was perforated, and is placed in the vicinity of the surgical bed.

The present disclosure was made in consideration of the above-described circumstances, and provides a robotically-assisted surgical system, a robotically-assisted surgical method, and a computer-readable medium that can assist in perforation of a port, which is performed after the placement of a surgical robot.

### SUMMARY

A robotically-assisted surgical system of a first aspect of the present disclosure that assists robotic surgery by a surgical robot having a robot main body includes one or more processors. The one or more processors are configured to plan a position of a port to be perforated on a body surface of a subject which is a target of the robotic surgery, acquire a captured image obtained by capturing the subject including at least a part of the subject by an overview camera included in the robot main body, recognize a planned position of the port in the captured image based on the captured image and the planned position of the port, and show the captured image and port position information indicating the planned position of the port in the subject illustrated in the captured image, on a display unit.

A robotically-assisted surgical method of a second aspect of the present disclosure that assists robotic surgery by a surgical robot having a robot main body includes: planning a position of a port to be perforated on a body surface of a subject which is a target of the robotic surgery; acquiring a captured image obtained by capturing the subject including at least a part of the subject by an overview camera included in the robot main body; recognizing a planned position of the port in the captured image based on the captured image and the planned position of the port; and showing the captured image and port position information indicating the planned position of the port in the subject illustrated in the captured image, on a display unit.

A non-transitory computer-readable medium of a third aspect of the present disclosure stores a program for causing a computer to execute a process. The process includes: planning a position of a port to be perforated on a body surface of a subject which is a target of robotic surgery by a surgical robot having a robot main body; acquiring a captured image obtained by capturing the subject including at least a part of the subject by an overview camera included in the robot main body; recognizing a planned position of the port in the captured image based on the captured image and the planned position of the port; and showing the captured image and port position information indicating the planned position of the port in the subject illustrated in the captured image, on a display unit.

The present disclosure has been made in consideration of the above-described circumstances and can assist in perforation of the port, which is performed after the placement of the surgical robot.

### BRIEF DESCRIPTION OF DRAWINGS

Exemplary embodiment(s) of the present invention will be described in detail based on the following figures, wherein:

FIG. 1 is a block diagram illustrating a configuration example of a robotically-assisted surgical system according to a first embodiment;

FIG. 2 is a block diagram illustrating a hardware configuration example of a robotically-assisted surgical device:

FIG. 3 is a block diagram illustrating a functional configuration example of the robotically-assisted surgical device:

FIG. 4 is a block diagram illustrating an electrical configuration example of a surgical robot:

FIG. 5 is a schematic view illustrating a structure example of the surgical robot;

FIG. 6 is a schematic view illustrating a first example of a placed posture of a robot main body:

FIG. 7 is a schematic view illustrating a second example of the placed posture of the robot main body;

FIG. 8 is a schematic view illustrating a first example of a port perforating posture of the robot main body;

FIG. 9 is a schematic view illustrating a second example of the port perforating posture of the robot main body;

FIG. 10 is a schematic view illustrating a first example of an equipped posture of the robot main body;

FIG. 11 is a schematic view illustrating a second example of the equipped posture of the robot main body;

FIG. 12 is a view illustrating an example of a state of a trocar, a surgical instrument, and the inside of a subject during robotic surgery:

FIG. 13 is a flowchart illustrating a generation example of a surgical plan by the robotically-assisted surgical device;

FIG. 14 is a view illustrating an example of a working area of the inside of the subject;

FIG. 15 is a flowchart illustrating an operation example during the robotic surgery by the surgical robot:

FIG. 16 is a schematic view illustrating an approaching example of the robot main body to a surgical bed:

FIG. 17 is a schematic view illustrating a placement example of the robot main body at a planned position in the vicinity of the surgical bed;

FIG. 18 is a schematic view illustrating an example of the robot main body in the port perforating posture at the planned position of the robot main body;

FIG. 19 is a schematic view illustrating an example of a landmark of the subject;

FIG. 20 is a schematic view illustrating a display example in which the landmark of the subject and the planned position of a port are superimposed on a body surface image of the subject;

FIG. 21 is a schematic view illustrating a display example in which the landmark of the subject and the planned position of the port are superimposed on a three-dimensional image of the subject;

FIG. 22 is a schematic view illustrating an example of the landmark of the subject included in an overview image and the planned position of the port:

FIG. 23 is a schematic view illustrating an example of the landmark of the subject included in the overview image, the planned position of the port, and port tolerance information;

FIG. 24 is a flowchart illustrating an operation example related to port registration by the robotically-assisted surgical system;

FIG. 25 is a flowchart illustrating an operation example related to the port registration by the robotically-assisted surgical system (continued from FIG. 24);

FIG. 26 is a view illustrating a superimposed display example of an overview image in which a perforating instrument is reflected and port position information;

FIG. 27 is a view illustrating a display example of guidance information for guiding the perforating instrument to the planned position of the port; and

FIG. 28 is a flowchart illustrating an operation example of a case of calculating a port position score by the robotically-assisted surgical device.

### DESCRIPTION OF EMBODIMENTS

Hereinafter, embodiments of the present disclosure will be described with reference to the drawings.

### First Embodiment

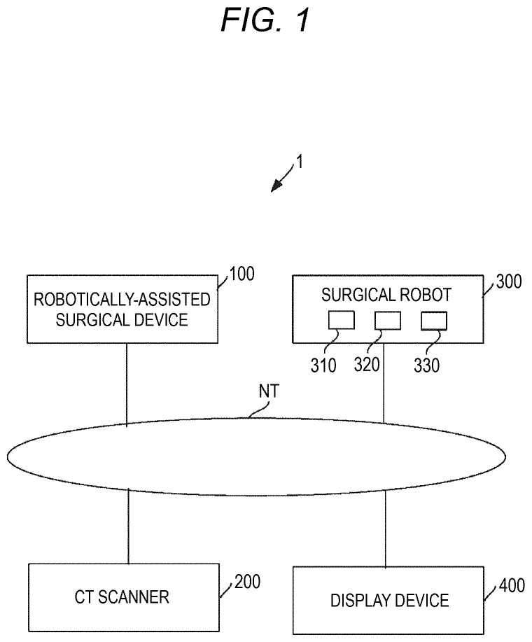

FIG. 1 is a block diagram illustrating a configuration example of a robotically-assisted surgical system 1 according to a first embodiment. The robotically-assisted surgical system 1 includes a robotically-assisted surgical device 100, a CT scanner 200, a surgical robot 300, and a display device 400. In FIG. 1, the robotically-assisted surgical device 100, the CT scanner 200, the surgical robot 300, and the display device 400 may be connected to each other via a network NT. The robotically-assisted surgical device 100, the CT scanner 200, the surgical robot 300, and the display device 400 may be connected to each other on a one-to-one or one-to-many basis without the network NT. The robotically-assisted surgical device 100, the CT scanner 200, the surgical robot 300, and the display device 400 may be temporarily separated from the connection with the network NT. The robotically-assisted surgical device 100 may be built into the surgical robot 300.

The robotically-assisted surgical device 100 acquires various pieces of data from the CT scanner 200 and the surgical robot 300. The robotically-assisted surgical device 100 performs image processing based on the acquired data to assist the robotic surgery by the surgical robot 300. The robotically-assisted surgical device 100 may be configured of a PC and software installed in the PC. The robotically-assisted surgical device 100 performs surgery navigation. The surgery navigation includes, for example, preoperative simulation for performing planning before surgery (preoperative planning) and intraoperative navigation for performing the assistance during surgery. The intraoperative navigation may be performed by the surgical robot 300.

The CT scanner 200 irradiates the subject with X-rays, and captures images (CT images) by using the difference in X-ray absorption by tissues in the body. The subject may include a living body, a human body, an animal, and the like. The CT scanner 200 generates the volume data including information on any location on the inside of the subject. The CT scanner 200 transmits the volume data as the CT image to the robotically-assisted surgical device 100 via a wired circuit or a wireless circuit. Imaging conditions for CT images or contrast conditions for administration of a contrast medium may be taken into consideration when capturing CT images.

The surgical robot 300 includes a robot operation terminal 310, a robot main body 320, and an image display terminal 330.

The robot operation terminal 310 includes a hand controller and a foot switch operated by an operator. The robot operation terminal 310 operates a plurality of robot arms AR provided in the robot main body 320 in response to the operation of the hand controller or the foot switch by the operator. The robot operation terminal 310 includes a viewer. The plurality of robot operation terminals 310 may exist, and the robotic surgery may be performed by a plurality of operators operating the plurality of robot operation terminals 310.

The robot main body 320 includes the plurality of robot arms AR for performing the robotic surgery, an end effector EF (forceps) attached to the robot arm AR, and the endoscope ES attached to the robot arm AR. Since the end effector EF and the endoscope ES are used for endoscopic surgery, the end effector EF and the endoscope ES are also referred to as surgical instruments 30 in the embodiment. The surgical instrument 30 includes at least one of one or more end effectors EF and endoscopes ES.

The robot main body 320 is provided with, for example, four robot arms AR, and includes a camera arm to which the endoscope ES is attached, a first end effector arm to which the end effector EF operated by the hand controller for the right hand of the robot operation terminal 310 is attached, a second end effector arm to which the end effector EF operated by the hand controller for the left hand of the robot operation terminal 310 is attached, and a third end effector arm to which the end effector EF for the replacement is attached. The robot main body 320, including the robot arm AR, may have a plurality of joints and may be equipped with a motor (an example of an actuator) and an encoder (an example of a sensor) corresponding to each joint. The encoder may include a rotary encoder as an example of an angle detector. Each robot arm AR has at least 6 degrees of freedom, preferably 7 or 8 degrees of freedom, and may operate in the three-dimensional space and be movable in each direction within the three-dimensional space. The end effector EF is an instrument that actually comes into contact with the treatment target in a subject PS in the robotic surgery, and enables various treatments (for example, grasping, excision, peeling, and suturing).

The end effector EF may include, for example, grasping forceps, peeling forceps, an electric knife, and the like. As the end effector EF, a plurality of separate end effector EFs different for each role may be prepared. For example, in the robotic surgery, the tissue may be suppressed or pulled by two end effector EFs, and the tissue may be cut by one end effector EF. The robot arm AR and the surgical instrument 30 may operate based on an instruction from the robot operation terminal 310. At least two end effectors EF are used in the robotic surgery.

A specific structure example of the robot main body 320 will be described later.

The image display terminal 330 has a monitor and a controller for processing the image captured by the endoscope ES and displaying the image on a viewer or a monitor. The monitor is confirmed, for example, by a robotic surgery assistant, a nurse, a radiologist, a student and the like (also referred to as assistant or the like).

The surgical robot 300 performs the robotic surgery in which an operation of the hand controller or the foot switch of the robot operation terminal 310 by the operator is received, the operations of the robot arm AR, the end effector EF, and the endoscope ES of the robot main body 320 are controlled, and various treatments for the subject PS are performed. The robotic surgery may be minimally invasive surgery and may be endoscopic surgery.

In the robotic surgery, a port PT may be perforated into the body surface of the subject PS, and pneumoperitoneum may be performed through the port PT. The port PT is a hole portion perforated into the subject PS. In the pneumoperitoneum, carbon dioxide may be delivered to inflate the abdominal cavity of the subject PS. A trocar TC may be installed at the port PT. The trocar TC has a valve and maintains an airtight state of the inside the subject PS. Air (for example, carbon dioxide) is continuously introduced into the subject PS for maintaining the airtight state.

The end effector EF (the shaft of the end effector EF) is inserted into the trocar TC. The valve of the trocar TC is opened when the end effector EF is inserted, and the valve of the trocar TC is closed when the end effector EF is detached. The end effector EF is inserted from the port PT via the trocar TC, and various treatments are performed depending on the surgical procedure. The surgical procedure indicates a method of surgery for the subject PS. In addition to laparoscopic surgery of which the surgery target is the abdomen, the robotic surgery may also be applied to endoscopic surgery which includes areas other than the abdomen as the surgery target.

The display device 400 may include, for example, an LCD or an organic EL. The display device 400 may be installed in the operating room, for example, on a wall, may be placed being suspended from the ceiling, or may be mounted on a moving device and placed at any position. The display device 400 receives various information and images from, for example, the robotically-assisted surgical device 100 and the surgical robot, and shows the various information and images. The display device 400 may be the image display terminal 330.

The robot main body 320 is placed outside the operating room before the robotic surgery, and is moved and placed inside the operating room from outside the operating room during the robotic surgery. Each device (for example, the robot operation terminal 310 and the image display terminal 330) of the surgical robot 300 other than the robot main body 320 may be installed in the operating room before the robotic surgery or may be moved and placed in the operating room from outside the operating room during the robotic surgery.

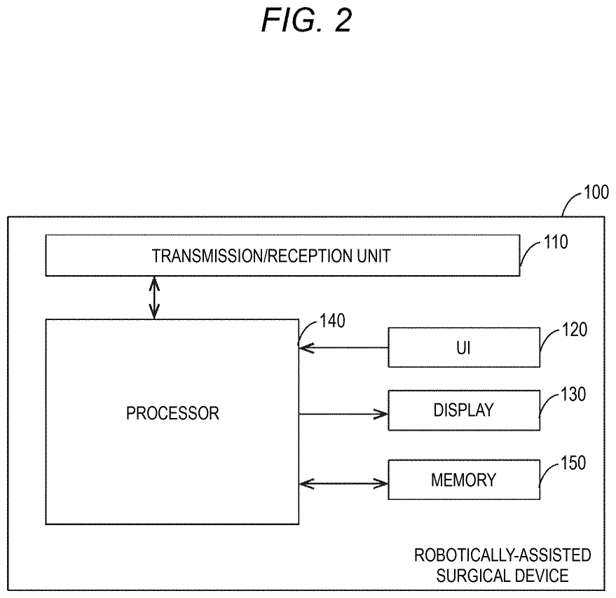

FIG. 2 is a block diagram illustrating a hardware configuration example of the robotically-assisted surgical device 100. The robotically-assisted surgical device 100 includes a transmission/reception unit 110, a UI 120, a display 130, a processor 140, and a memory 150.

The transmission/reception unit 110 includes a communication port, an external device connection port, a connection port to an embedded device, and the like. The transmission/reception unit 110 acquires various pieces of data from the CT scanner 200 and the surgical robot 300. The various pieces of acquired data may be immediately sent to the processor 140 (processing unit 160) for various types of processing, or may be sent to the processor 140 for various types of processing when necessary after being stored in the memory 150. The various pieces of data may be acquired via a recording medium or a storage medium.

The transmission/reception unit 110 transmits various pieces of data to the CT scanner 200 and the surgical robot 300. The various pieces of data to be transmitted may be directly transmitted from the processor 140 (the processing unit 160), or may be transmitted to each device when necessary after being stored in the memory 150. The various pieces of data may be sent via a recording medium or a storage medium.

The transmission/reception unit 110 may acquire volume data from the CT scanner 200. The volume data may be acquired in the form of intermediate data, compressed data or sinogram. The volume data may be acquired from information from a sensor device attached to the robotically-assisted surgical device 100. The volume data captured by the CT scanner 200 may be sent from the CT scanner 200 to an image data server (PACS: Picture Archiving and Communication Systems) and stored. The transmission/reception unit 110 may acquire the volume data from this image data server instead of acquiring the volume data from the CT scanner 200.

The transmission/reception unit 110 acquires information from the surgical robot 300. The information from the surgical robot 300 may include information on the kinematics of the surgical robot 300 (specifically, the robot main body 320). The information on the kinematics may include, for example, shape information regarding the shape and motion information regarding the motion of an instrument (for example, the robot arm AR, the end effector EF, the endoscope ES) for performing the robotic surgery included in the robot main body 320. The information on the kinematics may be received from an external server.

The shape information may include at least a part of information such as the length and weight of each part of the robot arm AR, the end effector EF, and the endoscope ES, the angle of the robot arm AR with respect to the reference direction (for example, a horizontal surface), and the attachment angle of the end effector EF with respect to the robot arm AR.

The motion information may include the movable range in the three-dimensional space of the robot arm AR, the end effector EF, and the endoscope ES. The motion information may include information such as the position, speed, acceleration, or orientation of the robot arm AR when the robot arm AR operates. The motion information may include information such as the position, speed, acceleration, or orientation of the end effector EF with respect to the robot arm AR when the end effector EF operates. The motion information may include information such as the position, speed, acceleration, or orientation of the endoscope ES with respect to the robot arm AR when the endoscope ES operates.

In the kinematics, together with the movable range of the robot arm itself, the movable range of the other robot arm is defined. Therefore, as the surgical robot 300 operates each robot arm AR of the robot main body 320 based on the kinematics, it is possible to avoid interference of the plurality of robot arms AR with each other during surgery.

An angle sensor may be attached to the robot arm AR, the end effector EF, or the endoscope ES. The angle sensor may include a rotary encoder that detects an angle corresponding to the orientation of the robot arm AR, the end effector EF, or the endoscope ES in a three-dimensional space. The transmission/reception unit 110 may acquire the detection information detected by various sensors attached to the surgical robot 300.

The transmission/reception unit 110 may acquire operation information regarding the operation with respect to the robot operation terminal 310. The operation information may include information such as an operation target (for example, the robot arm AR, the end effector EF, the endoscope ES), an operation type (for example, movement, rotation), an operation position, and an operation speed.

The information from the surgical robot 300 may include information regarding the imaging by the endoscope ES (endoscopic information). The endoscopic information may include an image captured by the endoscope ES (actual endoscopic image) and additional information regarding the actual endoscopic image (imaging position, imaging orientation, imaging viewing angle, imaging range, imaging time, and the like).

The UI 120 may include, for example, a touch panel, a pointing device, a keyboard, or a microphone. The UI 120 receives any input operation from the user of the robotically-assisted surgical device 100. Users may include operators, doctors, assistants, nurses, radiologists, students, and the like.

The UI 120 receives various operations. For example, an operation, such as designation of a region of interest (ROI) or setting of a brightness condition (for example, window width (WW) or window level (WL)), in the volume data or in an image (for example, a three-dimensional image or a two-dimensional image which will be described later) based on the volume data, is received. The ROI may include regions of various tissues (for example, blood vessels, organs, viscera, bones, and brain). The tissues may include diseased tissue, normal tissue, tumor tissue. Organs may also include the heart, lungs, liver, brain, and the like.

The display 130 may include an LCD, for example, and displays various pieces of information. The various pieces of information may include a three-dimensional image and a two-dimensional image obtained from the volume data. The three-dimensional images may include a volume rendering image, a surface rendering image, a virtual endoscopic image, a virtual ultrasound image, a CPR image, and the like. The volume rendering images may include a RaySum image, an MIP image, an MinIP image, an average value image, a raycast image, and the like. The two-dimensional images may include an axial image, a sagittal image, a coronal image, an MPR image, and the like.

The memory 150 includes various primary storage devices such as ROM and RAM. The memory 150 may include a secondary storage device such as HDD or SSD. The memory 150 may include a tertiary storage device such as a USB memory, an SD card, or an optical disk. The memory 150 stores various pieces of information and programs. The various pieces of information may include volume data acquired by the transmission/reception unit 110, images generated by the processor 140, setting information set by the processor 140, and various programs. The memory 150 is an example of a non-transitory recording medium in which a program is recorded.

The processor 140 may include, for example, a CPU, a DSP, or a GPU. The processor 140 functions as the processing unit 160 that performs various types of processing and controls by executing the program stored in the memory 150.

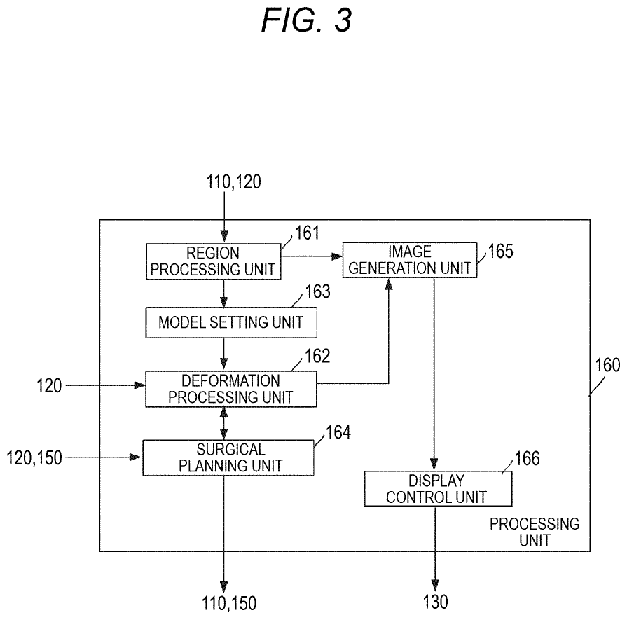

FIG. 3 is a block diagram illustrating a functional configuration example of the processing unit 160.

The processing unit 160 includes a region processing unit 161, a deformation processing unit 162, a model setting unit 163, a surgical planning unit 164, an image generation unit 165, a display control unit 166. The processing unit 160 controls each unit of the robotically-assisted surgical device 100. Each unit included in the processing unit 160 may be realized as different functions by one piece of hardware, or may be realized as different functions by a plurality of pieces of hardware. Each unit included in the processing unit 160 may be realized by a dedicated hardware component.

The region processing unit 161 acquires the volume data of the subject PS via the transmission/reception unit 110, for example. The region processing unit 161 extracts any region included in the volume data. The region processing unit 161 may automatically designate the ROI and extract the ROI based on a pixel value of the volume data, for example. The region processing unit 161 may manually designate the ROI and extract the ROI via the UI 120, for example. The ROI may be segmented (divided) and extracted including not only a single tissue but also tissues around the tissue. The tissue and the tissue neighbor thereof may be obtained by segmentation as separate tissues.

The model setting unit 163 sets a model of the tissue. The model may be set based on the ROI and the volume data. The model visualizes the tissue visualized by the volume data in a simpler manner than the volume data. Therefore, the data amount of the model is smaller than the data amount of the volume data corresponding to the model. The model is a target of deformation processing and deforming operation imitating various treatments in surgery, for example. The model may be, for example, a simple bone deformation model. In this case, the model deforms the bone by assuming a frame in a simple finite element and moving the vertices of the finite element. The deformation of the tissue can be visualized by following the deformation of the bone. The model may include an organ model imitating an organ (for example, rectum). The model may have a shape similar to a simple polygon (for example, a triangle), or may have other shapes. The model may be, for example, a contour line of the volume data indicating an organ. The model may be a three-dimensional model or a two-dimensional model. The bone may be visualized by the deformation of the volume data instead of the deformation of the model. This is because, since the bone has a low degree of freedom of deformation, visualization is possible by affine deformation of the volume data.

The model setting unit 163 may acquire the model by generating the model based on the volume data. A plurality of model templates may be predetermined and stored in the memory 150 or an external server. The model setting unit 163 may acquire a model by acquiring one model template among a plurality of model templates prepared in advance from the memory 150 or the external server in accordance with the volume data.

The volume data and the model are examples of 3D data (three-dimensional data) that three-dimensionally illustrates the state of the inside of the subject. In the embodiment, although the description may use either the volume data or the model as an example of the 3D data, the embodiment can also be applied to one of the volume data or the model, or the other one. The 3D data can be either data of a pneumoperitoneum state or data of a non-pneumoperitoneum data.

The deformation processing unit 162 performs processing related to the deformation in the subject PS which is a surgery target. For example, the tissue of an organ or the like in the subject PS can be subjected to various deforming operations by the user by imitating various treatments performed by the operator in surgery. The deforming operation may include an operation of lifting an organ, an operation of flipping an organ, an operation of cutting an organ, and the like. In response to this, the deformation processing unit 162 deforms the model corresponding to the tissue of an organ or the like in the subject PS. For example, an organ can be pulled, pushed, or cut by the end effector EF, but may be simulated by deforming the model in this manner. When the model deforms, the targets in the model may also deform. The deformation of the model may include movement or rotation of the model.

The deformation by the deforming operation may be performed with respect to the model and may be a large deformation simulation using the finite element method. For example, movement of an organ due to the body position change may be simulated. In this case, the elastic force applied to the contact point of the organ or the disease, the rigidity of the organ or the disease, and other physical characteristics may be taken into consideration. In the deformation processing with respect to the model, the computation amount is reduced as compared with the deformation processing with respect to the volume data. This is because the number of elements in the deformation simulation is reduced. The deformation processing with respect to the model may not be performed, and the deformation processing may be directly performed with respect to the volume data. In this case, the robotically-assisted surgical device 100 may not include the model setting unit 163.

The deformation processing unit 162 may virtually perform a pneumoperitoneum simulation for pneumoperitoneum on the subject PS, for example, as processing related to the deformation. A specific method of the pneumoperitoneum simulation may be a known method, for example, the method described in Reference Non-patent Literature 1 (Takayuki Kitasaka, Kensaku Mori, Yuichiro Hayashi, Yasuhito Suenaga, Makoto Hashizume, and Junichiro Toriwaki. “Virtual Pneumoperitoneum for Generating Virtual Laparoscopic Views Based on Volumetric Deformation”. MICCAI (Medical Image Computing and Computer-Assisted Intervention), 2004, P559-P567). In other words, the deformation processing unit 162 may perform the pneumoperitoneum simulation based on the volume data in the non-pneumoperitoneum state and generate the volume data in the virtual pneumoperitoneum state. The volume data obtained by capturing an image by the CT scanner 200 after the pneumoperitoneum is actually performed may also be used. The pneumoperitoneum simulation with changing pneumoperitoneum amount may be performed based on the volume data obtained by capturing an image by the CT scanner 200 after the pneumoperitoneum is actually performed. With the pneumoperitoneum simulation, the user can observe the virtual pneumoperitoneum state by assuming the pneumoperitoneum state of the subject PS even when there is no actual pneumoperitoneum in the subject PS. Among the pneumoperitoneum states, a state of pneumoperitoneum estimated by the pneumoperitoneum simulation may be referred to as a virtual pneumoperitoneum state, and a state of actual pneumoperitoneum may be referred to as an actual pneumoperitoneum state. A state where the pneumoperitoneum is not performed by the pneumoperitoneum simulation may be referred to as non-pneumoperitoneum state.

The pneumoperitoneum simulation may be a large deformation simulation using the finite element method. In this case, the deformation processing unit 162 may segment the body surface including the subcutaneous fat of the subject PS and the abdominal internal organs of the subject PS. Then, the deformation processing unit 162 may model the body surface as a two-layer finite element of skin and body fat, and model the abdominal internal organs as a finite element. The deformation processing unit 162 may segment, for example, lungs and bones in any manner and add the segmented result to the model. A gas region may be provided between the body surface and the abdominal internal organs, and the gas region (pneumopentoneum space) may be expanded (swollen) in accordance with virtual gas injection. The pneumoperitoneum simulation may not be performed.

The surgical planning unit 164 generates a surgical plan. The surgical plan may include planning the placement position of the robot main body 320, planning the moving route for deriving tolerances for the placement of the robot main body 320 at the position which is planned (planned position) and moving the robot main body 320 to the planned position of the robot main body 320, and the like. The surgical plan may include planning the posture of the robot main body 320, planning the posture of an arm base AB of the robot main body 320, and the like. The surgical plan may include planning the port position to be perforated on the body surface of the subject PS, generating the tolerance information on the placement of the port at the planned position, and the like. The surgical plan may include information on any landmark on the subject PS, information on the relative positional relationship between the landmark and the planned positions of each port, and the like.

The surgical planning unit 164 may determine the planned position of the robot main body 320, for example, based on the surgical procedure. For example, in a case where the surgical procedure is Transanal Minimally Invasive Surgery (TAMIS), the planned position of the robot main body 320 may be one of the positions neighbor of the foot side of the subject PS in the body axis direction. In a case where the surgical procedure is lung surgery, the subject PS may be in a lateral recumbent position against a surgical bed BD, and the planned position of the robot main body 320 may be in a left-right direction (that is, next to the surgical bed BD) perpendicular to the body axis direction of the subject PS. The surgical planning unit 164 may also determine the planned position of the robot main body 320 based on the 3D data of the subject PS and the kinematics of the robot main body 320.

The planned posture of the robot main body 320 can be a posture planned for each type of posture of the robot main body 320 (for example, placed posture, port perforating posture, equipped posture). The planned posture of the robot main body 320, for example, may be a posture defined by the combination of the postures (positions and orientations) of each member of the robot main body 320 in a case where the robot main body 320 takes a predetermined posture (for example, equipped posture).

The surgical planning unit 164 acquires information on a plurality of ports PT provided on the body surface of the subject PS. The information on the port PT may include the identification information of the port PT, the position information (port position) on the body surface of the subject PS where the port PT is perforated, the size information of the port PT, and the like. The information on the plurality of ports PT may be held in the memory 150 or an external server as a template. The information on the plurality of ports PT may be defined by the surgical procedure.

The surgical planning unit 164 may acquire the information on the plurality of port positions from the memory 150. The surgical planning unit 164 may acquire the information on the plurality of port positions from the external server via the transmission/reception unit 110. The surgical planning unit 164 may receive the designation of the port positions of the plurality of ports PT via the UI 120 and acquire the information on the plurality of port positions. The information on the plurality of port positions may be the information of a combination of the plurality of port positions. The port position may be considered as the planned position of the port PT as it is, or the planned position of the port PT may be derived based on the port position.

The surgical planning unit 164 may set the position of a target TG in the tissue (for example, liver) of the subject PS included in the volume data. The target TG is set in any tissue. The target TG is a target to be treated by the robotic surgery. The surgical planning unit 164 may designate the position of the target TG via the UI 120. The position of the target TG (for example, affected part) treated in the past for the subject PS may be stored in the memory 150. The surgical planning unit 164 may acquire and set the position of the target TG from the memory 150. The surgical planning unit 164 may set the position of the target TG depending on the surgical procedure. The target position may be a position of the region (target region) of the target TG having a certain width.

The surgical procedure may be designated via the UI 120. Each treatment in the robotic surgery may be determined by the surgical procedure. Depending on the treatment, the end effector EF required for the treatment may be determined. Accordingly, the end effector EF attached to the robot arm AR may be determined depending on the surgical procedure, and it may be determined which type of end effector EF is attached to which robot arm AR.

The surgical planning unit 164 may execute a surgical simulation. The surgical simulation can be a simulation for determining whether or not the desired robotic surgery in the subject PS is possible by the user operating the UI 120. In the surgical simulation, while assuming the surgery, by operating the end effector EF inserted from each port position in the virtual space, the user may determine whether or not the end effector EF can access the region of the target TG which is the surgery target. In other words, in the surgical simulation, it may be determined whether or not a movable unit (for example, the robot arm AR or the surgical instrument 30) related to the robotic surgery of the robot main body 320 can access the region of the target TG which is the surgery target without any problems, while receiving a manual operation of the UI 120 by the user.

In the surgical simulation, it may be determined whether or not the above-described access is possible based on the volume data of the subject PS, the combination of the plurality of port positions which were acquired, the kinematics of the robot main body 320, the surgical procedure, the volume data of the virtual pneumoperitoneum state, and the like. The surgical planning unit 164 may determine whether or not it is possible to access the target region at each port position while changing the plurality of port positions on the body surface of the subject PS, and may perform the surgical simulation sequentially. The surgical planning unit 164 may obtain information on the final preferred (for example, optimal) combination of port positions from the surgical simulation and use the information as the planned position for each port PT. In this manner, the robotically-assisted surgical device 100 can allow the user to manually adjust the planned position of the port PT and plan the port position.

The surgical planning unit 164 may also derive (for example, calculate) a surgical planning score that indicates the appropriateness of robotic surgery according to the surgical plan. The surgical planning score may be derived based on at least one of the planned position of the robot main body 320, the planned posture of the robot main body 320, and the planned position of the port PT. For example, the appropriateness for the robotic surgery performed using the surgical instrument 30 which is placed at the planned position of the robot main body 320, has the robot main body 320 in the planned posture, and is inserted through the port PT perforated at the planned position, may be indicated by a surgical planning score. In addition to the description above, the surgical planning score may also be calculated based on the planned posture of the arm base AB of the robot main body 320.

The surgical planning score indicates the value of the combination of each element in the surgical plan, such as the planned position of the robot main body 320, the planned posture of the robot main body 320, and the planned position of the port PT. The surgical planning unit 164 may generate a plurality of candidates for combinations of each element for the planned position of the robot main body 320, the planned posture of the robot main body 320, the planned position of the port PT, and the like. The surgical planning score may be derived for each candidate of the planned position of the robot main body 320. The surgical planning score may be derived for each candidate of the planned posture of the robot main body 320. The surgical planning score may be derived for each candidate of the planned position of the port PT. The surgical planning score may be derived for each candidate of the planned posture of the arm base AB. The derivation of the surgical planning score will be supplemented later.

The surgical planning score may include a port position score that indicates the ease of the robotic surgery via the planned position of the port PT. The port position score may be calculated based on the kinematics of the robot main body 320, the surgical procedure, the volume data of the virtual pneumoperitoneum state, and the like, as well as the combination of the plurality of port positions. The port position score may be derived based on at least one of the size of a working area WA, the type of treatment that can be performed at each position within the working area WA, and the movable range of the robot arm AR during surgery. The working area WA is a range in the subject PS that can be approached by the plurality of surgical instruments 30. The surgical planning unit 164 may calculate the port position score by weighting the location closer to the target TG in the working area WA.

The surgical planning score may also include an arm interference score that indicates the ease of interference between the plurality of robot arms AR included in the robot main body 320. For example, the higher the arm interference score, the more likely the robot arms AR interfere with each other, and the lower the arm interference score, the less likely the robot arms AR interfere with each other. When approaching any position, the robot arm AR can take a plurality of different postures, using the degree of freedom of the robot arm AR that exceeds 6. By switching between the plurality of different postures, interference between the robot arms AR can be avoided.

The arm interference score may be derived based on the movable range of the robot arm AR in the treatment that can be performed at any position in the working area WA. The arm interference score may further be derived based on the planned position of the robot main body 320 and the planned posture of the robot main body 320.

The surgical planning score may include a robot stability score that indicates the difficulty of falling of the robot main body 320. The robot stability score may be calculated based on the kinematics of the robot main body 320, the position of the center of gravity of the robot main body 320 in a case where the robot main body 320 is in the planned posture, the size of the space (corresponding to the perforating workspace) between the robot main body 320 and the subject PS, and the like.

The surgical planning unit 164 may calculate the surgical planning score based on at least one of the port position score, the arm interference score, and the robot stability score. For example, even in a case where the value of the port position score that does not take into account the planned position or planned posture of the robot main body 320 is the maximum, in a case where the interference between the robot arms AR is large or the stability of the robot main body 320 during the robotic surgery is insufficient, the planned positions of the plurality of ports PT corresponding to the port position score may be adopted in the surgical plan.

The surgical planning unit 164 may adjust the contents of the surgical plan (also referred to as surgical plan adjustment) based on the surgical planning score. For example, at least one of the planned position of the robot main body 320, the posture of the robot main body 320 and the arm base AB, the planned position of the port PT, and the posture of the arm base AB, may be adjusted. In this case, the surgical planning unit 164 may adjust the surgical plan based on the amount of variation in the surgical planning score due to changes in the surgical plan. Changes in the surgical plan include moving the planned position of the robot main body 320, changing the planned posture of the robot main body 320 and the arm base AB, moving the planned position of the port PT, and the like. The surgical plan adjustment will be supplemented later.

In this manner, the surgical planning unit 164 may derive the plurality of port positions, which is a perforation target, according to the surgical simulation. The surgical planning unit 164 may develop a surgical plan based on the surgical planning score.

The surgical planning unit 164 calculates the tolerance of the planned position of the port PT and generates the tolerance information indicating this tolerance. This tolerance indicates an error allowed in perforating with respect to the planned position of the port PT. The range indicating the tolerance is a range surrounding the planned position of the port PT. The surgical planning unit 164 may calculate the tolerance based on the surgical planning score. In this case, the tolerance may be calculated based on the amount of variation (decrease amount) in the surgical planning score due to the movement of the planned position of the port PT. For example, a range where the decrease amount of the surgical planning score from the surgical planning score (for example, the maximum value of the surgical planning score) at the planned position of the port PT is equal to or less than a threshold value th1 may be determined as a tolerance range of the planned position of the robot main body 320.

The surgical planning unit 164 may recognize landmarks of the subject PS based on the volume data of the subject PS and generate information on the landmarks. The landmarks of the subject PS are the parts of the subject PS that can be visually specified from the outside of the subject PS. There may be one or a plurality of landmarks. The surgical planning unit 164 may acquire the designation information for designating a specific landmark via the UI 120 and generate the information on the designated landmark. The information on the landmarks may include the type of landmark (for example, umbilical), a part of the landmark in the volume data, and the like.

The image generation unit 165 may generate a three-dimensional image or a two-dimensional image based on the volume data acquired by the transmission/reception unit 110. The image generation unit 165 may generate a three-dimensional image or a two-dimensional image based on the region of a part of the volume data extracted by the region processing unit 161. The three-dimensional image may include a body surface image illustrating the body surface of the subject PS.

The display control unit 166 causes the display 130 to display various types of data, information, and images. The display control unit 166 may display a three-dimensional image or a two-dimensional image generated by the image generation unit 165. The display control unit 166 may also adjust the brightness of the rendering image. The brightness adjustment may include, for example, adjustment of at least one of a window width (WW) and a window level (WL).

Next, a configuration example of the robot main body 320 of the surgical robot 300 will be described.

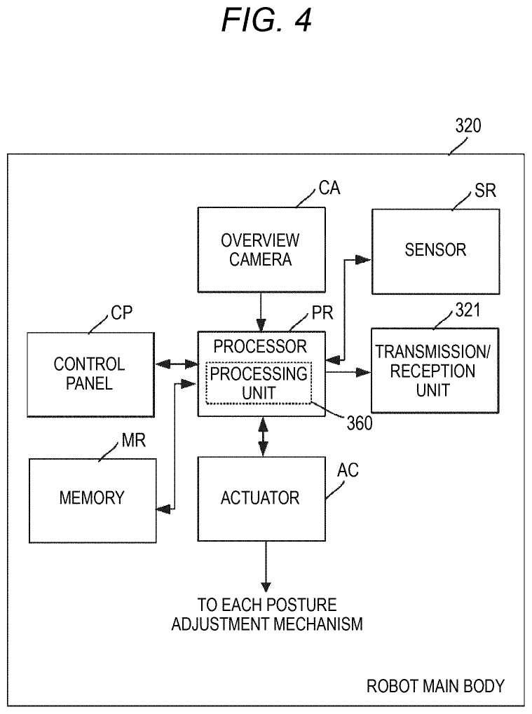

FIG. 4 is a block diagram illustrating an electrical configuration example of the robot main body 320. The robot main body 320 includes a processor PR, a transmission/reception unit 321, an overview camera CA, a sensor SR, an actuator AC, a control panel CP, and a memory MR.

The transmission/reception unit 321 includes a communication port, an external device connection port, a connection port to an embedded device, and the like. The transmission/reception unit 110 acquires various pieces of data from the robotically-assisted surgical device 100 and the CT scanner 200. The various pieces of acquired data may be immediately sent to the processor PR (processing unit 360) for various types of processing, or may be sent to the processor PR for various types of processing when necessary after being stored in the memory MR. The various pieces of data may be acquired via a recording medium or a storage medium.

The transmission/reception unit 110 transmits various pieces of data to the robotically-assisted surgical device 100 and the CT scanner 200. The various pieces of data to be transmitted may be directly transmitted from the processor PR, or may be transmitted to each device when necessary after being stored in the memory MR. The various pieces of data may be sent via a recording medium or a storage medium.

The processor PR may include, for example, an MPU, a CPU, or a DSP. The processor PR functions as the processing unit 360 that performs various types of processing and controls by executing the program stored in the memory MR.

The overview camera CA captures an image of the subject within the imaging range and obtains an overview image. In the overview image, a part or the whole of the subject PS is reflected, for example, w % ben the robot main body 320 enters the operating room or is placed at the planned position. In the overview image, a state of the inside of the operating room may be reflected.

The memory MR stores, for example, various pieces of data, information, or programs. The memory MR includes various primary storage devices such as ROM and RAM. The memory MR may include a secondary storage device such as HDD or SSD. The memory MR may include a tertiary storage device such as a USB memory, an SD card, or an optical disk.

The various pieces of information stored in the memory MR may include the captured image captured by the overview camera CA (also referred to as overview image), the information to be processed or processed by the processor PR, and the like. The information to be processed or processed by the processor PR may include, for example, information on the surgical plan of surgery by the robot main body 320. The memory MR is an example of a non-transitory recording medium in which a program is recorded.

The actuator AC provides a driving force to each posture adjustment mechanism for changing the posture of the robot main body 320 under the control of the processor PR. This posture adjustment mechanism may include, for example, a rotation mechanism, a slide mechanism, or an expansion/contraction mechanism. The posture adjustment mechanism may be included in each member of the robot main body 320, for example, or in a joint JT that connects each member to each other. The robot main body 320 can change the posture of the robot main body 320 without manual intervention during surgery as the actuator AC provides a driving force to each posture adjustment mechanism at a desired timing.

The sensors SR include position detectors (for example, linear encoders), angle detectors (for example, rotary encoders), and the like. The sensor SR may iteratively detect the position and angle of each member in the robot main body 320, and detect the movement of each member in the robot main body 320. The detection result by the sensor SR is sent to the processor PR.

The control panel CP is configured with, for example, a touch panel and has a function as an operation unit and a display unit. The control panel CP receives various operations and sends the operation information to the processor PR. The various operations may include an operation to designate the posture of the whole or a part of the robot main body 320, an operation related to the movement of the robot main body 320, and the like. The designation of the posture of the robot main body 320 may include the designation of the type of posture of the robot main body 320. The control panel CP displays various pieces of information. For example, the information on the operation may be displayed, guide information and operation options for the operation may be displayed, or the results of the operation may be displayed. The control panel CP is operated by the user and the display is confirmed by the user. The operation unit and the display unit may be configured separately.

Next, the details of the processing unit 360 will be described.

The processing unit 360 controls each unit of the robot main body 320. The processing unit 360 controls the posture of the robot main body 320. The processing unit 360 may control the posture of the whole or a part of the robot main body 320 based on the designation information of the posture of the robot main body 320 from the control panel CP. The processing unit 360 may acquire the planned posture of the robot main body 320 from the memory MR and the like, and control the posture of the robot main body 320 based on the planned posture. In this case, the posture of the robot main body 320 may be controlled by controlling the provision of a driving force from the actuator AC to each posture adjustment mechanism. The posture of the robot main body 320 may be controlled based on the information detected by the sensor SR.

The processing unit 360 acquires the operation information from the robot operation terminal 310 via the transmission/reception unit 321, and controls the operation of the robot arm AR and the surgical instrument 30 based on this operation information.

The processing unit 360 may acquire the overview image captured by the overview camera CA. The processing unit 360 may acquire the information on the surgical plan from the robotically-assisted surgical device 100. The processing unit 360 may perform port registration based on the overview image and the surgical plan. The port registration is registration of the planned port position in the virtual space (in the 3D data) with the port position in the actual space. Specifically, the processing unit 360 may recognize the planned position (image position) of the port PT in the overview image obtained during the robotic surgery.

The processing unit 360 may perform the port registration based on the landmarks of the subject PS. In this case, the processing unit 360 recognizes landmarks by image analysis of the overview image. The processing unit 360 also recognizes the planned position of the port PT in an overview image G1 based on the recognized landmarks and the planned position of the port PT with respect to the landmarks included in the surgical plan. There may be one or a plurality of planned positions of the port PT in the overview image G1. The processing unit 360 may recognize the tolerance range (image range) with respect to the planned position of the port PT in the overview image based on the recognized landmarks, the planned position of the port PT with respect to the landmarks, and the tolerance information included in the surgical plan.

The processing unit 360 may display the port position information indicating the planned position of the port PT, corresponding to the planned position of the port PT recognized in the overview image. The processing unit 360 may show the tolerance information indicating the tolerance of the port PT, corresponding to the tolerance range of the port PT recognized in the overview image.

The tolerance information may be shown as graphic information or character information. The graphic information may be illustrated in a range that includes tolerance including the planned position of the port PT. This range can be a two-dimensional range on the body surface of the subject PS. The two-dimensional range may be a range indicated by a circle (ellipse, perfect circle, or other circle), polygon (for example, rectangle, square, triangle, or other polygon), or other shape. Circles and polygons are also referred to as primitive shapes. The tolerance information may be shown as other information (for example, information on the display mode (display color, display size, display pattern, and flashing pattern)). For example, in a case where the tolerance of the planned position of the port PT is large, the planned position of the port PT may be shown in a first color, and in a case where the tolerance is small, the planned position of the port PT may be shown in a second color.

As the robotically-assisted surgical system 1 displays the tolerance information, the assistant or others can visually recognize the tolerance information and can quickly grasp the extent to which the shift of the actual perforation position from the planned position of the port PT is allowable.

For example, in a case where the range indicated by the tolerance information is large, the assistant or others can recognize that the port PT can be perforated roughly. It is possible to reduce the psychological burden on the assistant or others. The robotically-assisted surgical system 1 may have somewhat low accuracy of the planned position of the port PT, can reduce the calculation and man-hours required for deriving the planned position of the port PT, and can shorten the time required for the surgical plan.

For example, in a case where the range indicated by the tolerance information is small, the assistant or others can recognize that the port PT needs to be perforated precisely at the planned position. The robotically-assisted surgical system 1 can alert the user that a high degree of accuracy is required when perforating the port PT at the planned position.

Next, the display device 400 will be described.

The operating room may be provided with a display device 400 separate from the display 130 of the robotically-assisted surgical device 100 or the control panel CP of the robot main body 320. There may be one or a plurality of display devices 400. The display device 400 may display a three-dimensional image or a two-dimensional image of the subject PS generated by the robotically-assisted surgical device 100. The display device 400 may display the actual endoscopic image of the inside of the subject PS captured during surgery. The display device 400 may display the overview image captured by the overview camera CA. The display device 400 may display information on the intraoperative navigation (for example, port position information, tolerance information, and various guidance information). The plurality of display devices may display the same or different images.

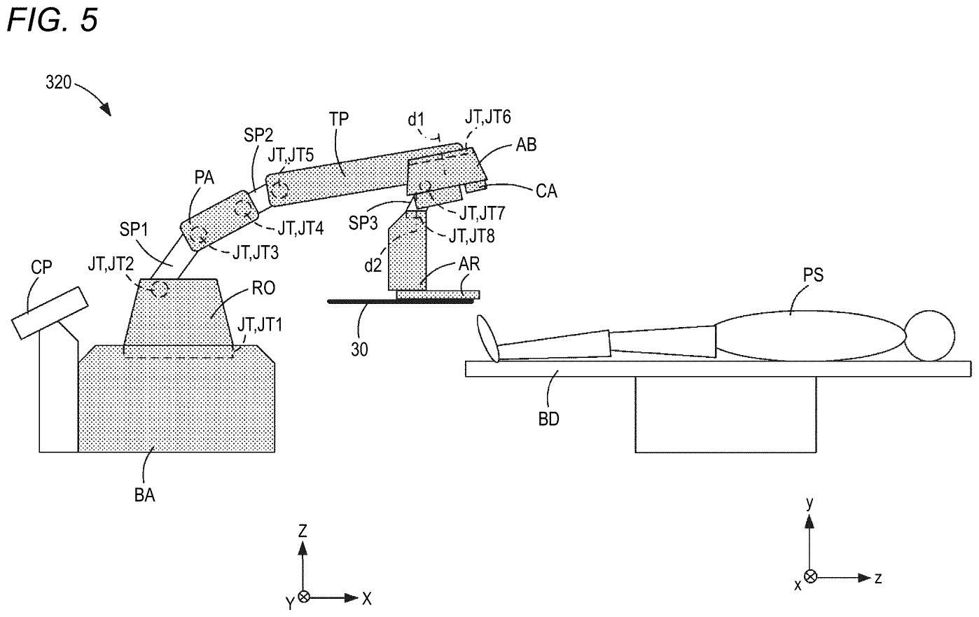

FIG. 5 is a view illustrating a structure example of the robot main body 320. Here, the description will be omitted or simplified regarding configurations that are similar to the electrical configuration of FIG. 4 described earlier.

In FIGS. 5 to 11, the coordinate system of the operating room is illustrated using XYZ. An X-direction is one direction along the floor surface of the operating room. AY-direction is a direction perpendicular to the X-direction along the floor surface of the operating room. A Z-direction is a direction perpendicular to the X-direction and Y-direction, that is, a direction along the vertical direction. The X-direction, the Y-direction, and the Z-direction may be other directions, and may not be based on the operating room.

In FIG. 5, the coordinate system of the subject PS is illustrated using xyz. The x-direction may be along the left-right direction with respect to the subject PS. The y-direction may be the front-rear direction (thickness direction of the subject PS) with respect to the subject PS. The z-direction may be an up-down direction (the body axial direction of the subject PS) with respect to the subject PS. The x-direction, the y-direction, and the z-direction may be three directions defined by digital imaging and communications in medicine (DICOM). The x-direction, the y-direction, and the z-direction may be other directions, and may not be based on the subject PS.

In FIG. 5, the X-direction matches the z-direction, the Y-direction matches the x-direction, and the Z-direction matches the y-direction. The orientation of the surgical bed BD and the subject PS with respect to the robot main body 320 is not limited thereto. Therefore, the relationship between the coordinate system of the operating room and the coordinate system of the subject PS is not limited to the above-described correspondence.

The robot main body 320 includes a base BA, the control panel CP, a rotating base RO, a parent arm PA, a ceiling member TP, the arm base AB, the overview camera CA, the robot arm AR, and the surgical instrument 30. The robot main body 320 is configured with support members SP1, SP2, and SP3 and joints JT (JT1, JT2, JT3, JT4, JT5, JT6, JT7, and JT8).

The joint JT is connected to at least one of the members included in the robot main body 320. The joint JT has a posture adjustment mechanism for adjusting the posture of the robot main body 320, the sensor SR that measures the state of the posture adjustment mechanism, and the actuator AC that provides a driving force to the posture adjustment mechanism. The posture adjustment mechanism may include a rotation mechanism in which one member rotates with respect to the other one member of the two members connected to the joint JT. The posture adjustment mechanism may include a slide mechanism in which one member connected to the joint JT moves in parallel. The posture adjustment mechanism may include the expansion/contraction mechanism in which one member connected to the joint JT expands and contracts. Accordingly, the posture adjustment mechanism can change the state of each member connected to the joint JT, or change the positional relationship of plurality of members connected to the joint JT.

The base BA is placed on the floor in the operating room during the robotic surgery. The placement position of the base BA may be the placement position of the robot main body 320. The base BA has members for the robot main body 320 to move, such as tires, lock members that regulate the movement of the robot main body 320, and the like, at the lower portion of the base BA. The placement position of the robot main body 320 may be the position where the entire robot main body 320 is projected onto the floor.

The rotating base RO is connected to the base BA via the joint JT1. The rotating base RO is rotatable with respect to the base BA, for example, along the XY plane with the Z-direction passing through the center of the joint JT1 on the XY plane as the rotation center.

The support member SP1 is connected to the rotating base RO via the joint JT2. The support member SP1 is rotatable with respect to the rotating base RO, for example, along the XY plane with the Y-direction passing through the center of the joint JT2 on the XY plane as the rotation center.

The parent arm PA is connected to the support member SP1 via the joint JT3. The parent arm PA is rotatable with respect to the support member SP1, for example, along the XZ plane with the Y-direction passing through the center of the joint JT3 on the XZ plane as the rotation center.

The support member SP2 is connected to the parent arm PA via the joint JT4. The support member SP2 is rotatable with respect to the parent arm PA, for example, along the XZ plane with the Y-direction passing through the center of the joint JT4 on the XZ plane as the rotation center.

The ceiling member TP is connected to the support member SP2 via the joint JT5. The ceiling member TP is rotatable with respect to the support member SP2, for example, along the XZ plane with the Y-direction passing through the center of the joint JT5 on the XZ plane as the rotation center.

The arm base AB is connected to the ceiling member TP via the joint JT6. The arm base AB is rotatable with respect to the ceiling member TP, for example, along the facing surface with a direction d1 (arrangement direction of the ceiling member TP and the arm base AB) perpendicular to the facing surface through the center of the joint JT6 on the facing surface of the ceiling member TP and the arm base AB as the rotation center.

The support member SP3 is connected to the arm base AB via the joint JT7. The support member SP3 is rotatable with respect to the arm base AB, for example, along the XZ plane with the Y-direction passing through the center of the joint JT7 on the XZ plane as the rotation center.

The robot arm AR is connected to the support member SP3 via the joint JT8. The robot arm AR is rotatable with respect to the support member SP3, for example, along the connection surface with a direction d2 perpendicular to the connection surface passing through the center of the joint JT8 on the connection surface between the robot arm AR and the support member SP3 as the rotation center.

The robot arm AR has a first part and a second part along the two directions. The two directions can be directions perpendicular to each other. In FIG. 5, the robot arm AR has the first part along the Z-direction and the second part along the X-direction. For example, when the joint JT7 or the joint JT8 rotates, the direction in which the two parts of the robot arm AR extend changes from the state illustrated in FIG. 5. Although not illustrated in the drawing, the robot arm AR is connected to multiple joints or has multiple joints, and has 8 degrees of freedom for the movement of the robot arm AR according to the movement of the multiple joints.

The surgical instrument 30 is connected to the second part of the robot arm AR. The second part of the robot arm AR has a slide mechanism that allows the surgical instruments 30 to be slidable along the second part. The surgical instrument 30 has 4 degrees of freedom for the movement of the surgical instrument 30. 4 degrees of freedom may include being movable in a direction along the second part of the robot arm AR, being rotatable with the extending direction of the surgical instrument 30 as the rotation center, being able to bend the distal end portion of the surgical instrument 30 to bow, being able to open and close the distal end portion of the surgical instrument 30, and the like.

The trocar TC is not directly attached (not connected) to the robot arm AR. Accordingly, the robot main body 320 can ensure a space between the trocar TC and the robot arm AR, and makes it easier to ensure a workspace when perforating the port PT or operating the surgical instrument 30 during surgery. The robot arm AR and the trocar TC are not connected to each other, and accordingly, the relative position between the robot arm AR and the rotation center of the surgical instrument 30 where the trocar TC is positioned is variable. Therefore, the robot main body 320 can realize the movement of the robot arm AR with an even higher degree of freedom.

In this manner, the robot main body 320 can flexibly adjust the posture of the whole or a part of the robot main body 320 by changing the posture (position and orientation) of the members connected to the joint JT, or by changing the relative positional relationship and orientation of the plurality of members connected to the joint JT. In particular, as the robot arm AR has 8 degrees of freedom, it is possible to move the robot arm AR without moving the surgical instrument 30, and to suppress interference between the robot arms AR. The position of the joint JT and the movement of each member due to the action of the joint JT are not limited to the contents described above. For example, at least one of the joints JT may have the slide mechanism or the expansion/contraction mechanism, and each member may slide or expand or contract, or each member of the robot main body 320 may rotate in a direction different from that illustrated in FIG. 5.

The robot main body 320 has the arm base AB installed at the distal end portion of the ceiling member TP. The arm base AB can be tilted with respect to the horizontal direction by the action of each joint JT provided in the robot main body 320. The robot main body 320 can easily ensure a space between the robot main body 320 and the subject PS, which is the target of robotic surgery, by tilting the arm base AB, and it is possible to improve operability during surgery. For example, the robot main body 320 can easily take the port perforating posture by the assistant or others.

Next, a specific example of the posture of the robot main body 320 will be described.

The robot main body 320 can take various postures during surgery. The posture of the robot main body 320 includes, for example, the placed posture, the port perforating posture, and the equipped posture. The robot main body 320 changes the posture of the robot main body 320, for example, in the order of the placed posture, the port perforating posture, and the equipped posture. After the equipped posture, the robot main body 320 will be in a posture corresponding to the actual surgical procedure, based on the operation of the operator via the robot operation terminal 310, for example.

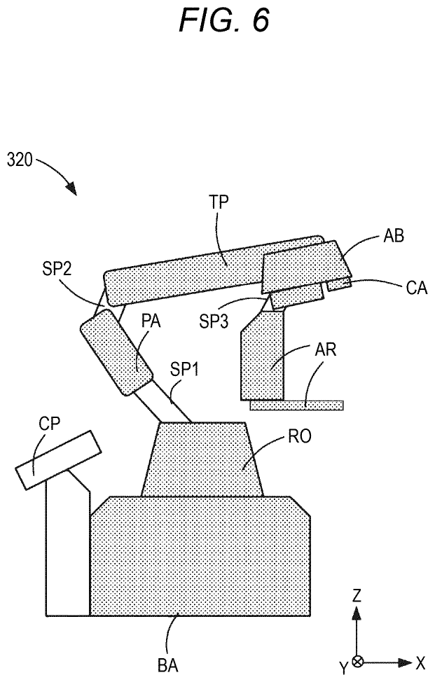

FIGS. 6 to 11 illustrate various postures of the robot main bodies 320 and 320A. The robot main body 320A has a support member SP4 instead of the parent arm PA and the support members SP1 and SP2 of the robot main body 320. The support member SP4 can expand and contract by the expansion/contraction mechanism of the joint JT. The other configurations of the robot main body 320A are the same as those of the robot main body 320, and thus, the description thereof will be omitted. Accordingly, the robot main body 320A is a modification example of the robot main body 320.

The placed posture is a posture of the robot main body 320 when being moved to the operating room and placed in the vicinity of the surgical bed BD on which the subject PS is placed. The placed posture is a posture in which the size (volume) on the space surrounded by each member of the robot main body 320 is equal to or less than a threshold value th2 (for example, minimum). The robot main body 320 takes the placed posture so as not to be in contact with for example, the door of the operating room, various devices installed in the operating room, and people in the operating room, or so as not to interfere with the movement (line of movement) of people in the operating room.

The surgical planning unit 164 of the robotically-assisted surgical device 100 calculates the planned posture related to the placed posture based on the kinematics of the robot main body 320, for example. A predetermined placed posture may be prepared in advance as a template, and this placed posture may be used as the planned posture. The processing unit 360 of the robot main body 320 acquires the planned posture related to the placed posture included in the surgical plan, controls the posture according to the planned posture, and takes the planned posture related to the placed posture.

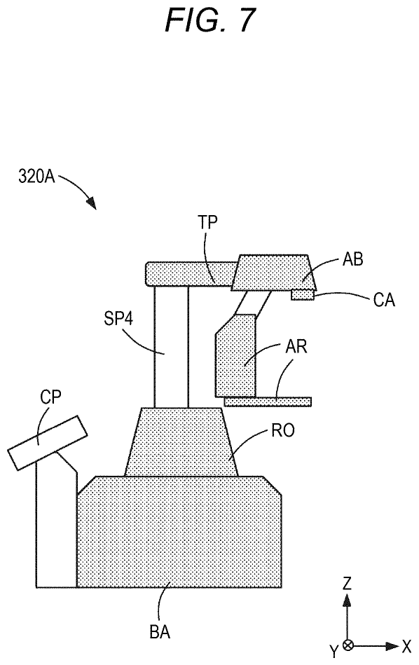

FIG. 6 is a schematic view illustrating an example of the placed posture of the robot main body 320. FIG. 7 is a schematic view illustrating an example of the placed posture of the robot main body 320A.

In FIG. 6, the robot arm AR is as close as possible to the rotating base RO, and the entire robot main body 320 is compact. In FIG. 7, the support member SP4 has been shrunk, and the entire robot main body 320A has become compact.

The port perforating posture is a posture when perforating the port PT into the subject PS. The port perforating posture is a posture that ensures as much space as possible around the subject PS. The port perforating posture can be, for example, a posture in which the size of the space (also referred to as perforating workspace) between the robot arm AR and the subject PS is equal to or greater than a threshold value th3 (for example, maximum). The robot main body 320 takes the port perforating posture, such that, for example, it is possible to suppress interference of the robot arm AR with the perforation work of the port PT by the assistant or the like. In order to achieve the port perforating posture, the joint JT that moves the parent arm PA and the rotating base RO may be driven, and the robot arm AR may be controlled to be as far away from the subject PS as possible. The processing unit 360 may actuate the robot arm AR, actuate the arm base AB, or actuate both the robot arm AR and the arm base AB such that the robot arm AR moves away from the subject PS in a case where the robot main body 320 is in the port perforating posture. As a result, the robot arm AR may be sufficiently far away from the subject PS.

The surgical planning unit 164 of the robotically-assisted surgical device 100 calculates the planned posture related to the port perforating posture based on the kinematics of the robot main body 320, for example. A predetermined port perforating posture may be prepared in advance as a template, and this port perforating posture may be used as the planned posture. The processing unit 360 of the robot main body 320 acquires the planned posture related to the port perforating posture included in the surgical plan, controls the posture according to the planned posture, and takes the planned posture related to the port perforating posture.

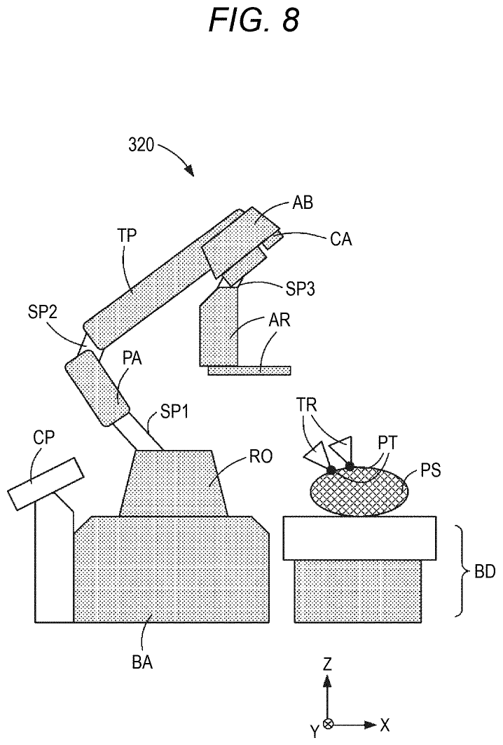

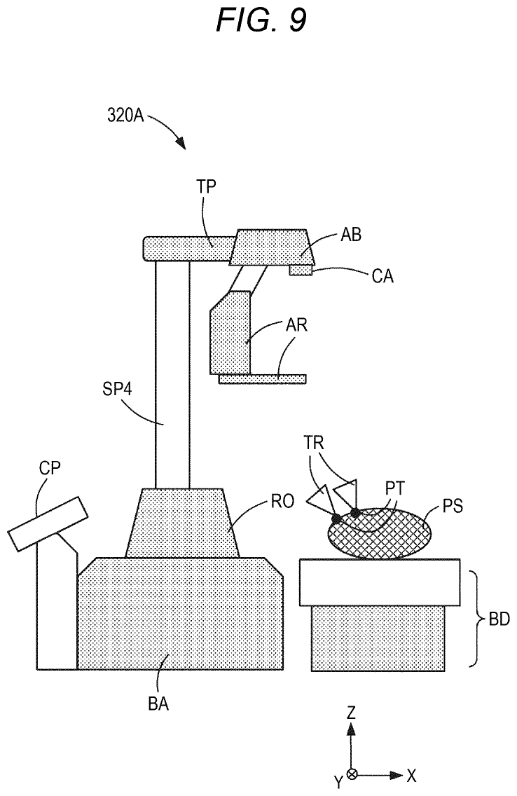

FIG. 8 is a schematic view illustrating an example of the port perforating posture of the robot main body 320. FIG. 9 is a schematic view illustrating an example of the port perforating posture of the robot main body 320A.

In FIG. 8, the rotating base RO and the arm base AB are as far away as possible, and a large perforating workspace is ensured. In FIG. 9, the support member SP4 is elongated, and a larger perforating workspace is ensured.

The equipped posture is a posture when the robot arm AR is equipped with the surgical instruments 30 and the like as the end effectors EF and the surgical instrument 30 is inserted into the subject PS passing through the perforated port PT In other words, the surgical instrument 30 is attached in the equipped posture and is not attached in the placed posture and the port perforating posture. The equipped posture is determined based on the internal state of the subject PS (for example, the position of each organ in the subject PS and the position of the target TG), the surgical procedure, and the like. The equipped posture may be, for example, a posture in which the movable range of the surgical instrument 30 during surgery, that is, the working area WA is equal to or greater than a threshold value th4 (for example, maximum). The equipped posture may be a posture in which the movable range of the robot arm AR during surgery is equal to or greater than a threshold value th42 (for example, maximum), that is, the arm interference score indicating a degree of interference between the robot arms is equal to or less than a threshold value th43 (for example, minimum) corresponding to the threshold value th42.

The surgical planning unit 164 of the robotically-assisted surgical device 100 may calculate the planned posture related to the equipped posture based on the kinematics of the robot main body 320, the volume data of the subject PS, the surgical procedure, and the like. A predetermined equipped posture may be prepared in advance as a template, and this equipped posture may be used as the planned posture. The processing unit 360 of the robot main body 320 acquires the planned posture related to the equipped posture included in the surgical plan, controls the posture according to the planned posture, and takes the planned posture related to the equipped posture.

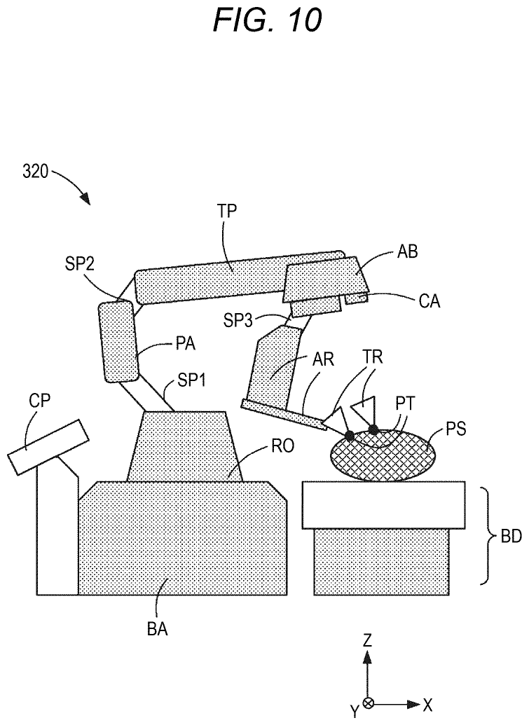

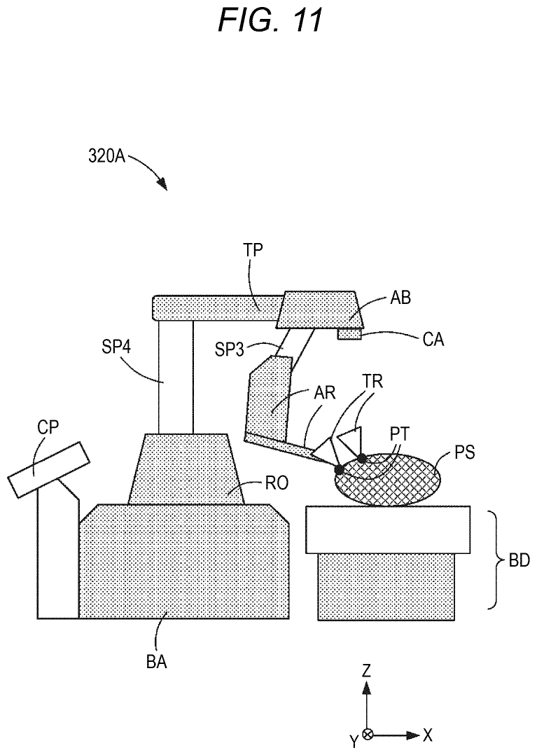

FIG. 10 is a schematic view illustrating an example of the equipped posture of the robot main body 320. FIG. 11 is a schematic view illustrating an example of the equipped posture of the robot main body 320A.

After the robot main body 320 is in the equipped posture, the operation for the treatment during surgery is performed. In this case, the robot main body 320 does not change the placement position of the robot main body 320, the posture of the parent arm PA, and the posture of the rotating base RO (does not change the mode).

In this manner, the robot main body 320 can enter the operating room in a compact posture as much as possible and be placed in the vicinity of the surgical bed BD. After entering the room, the robot main body 320 ensures a large perforating workspace and can perforate the port PT. Accordingly, compared to a case where the robot main body 320 is placed in the vicinity of the surgical bed BD after the port PT is perforated, the time from the perforation of the port PT to the completion of the surgery can be shortened, and the burden on the subject PS can be reduced. After the robot arm AR is equipped with the surgical instrument 30 and the surgical instrument 30 is inserted into the subject PS through the port PT, the necessary treatment can be performed smoothly.

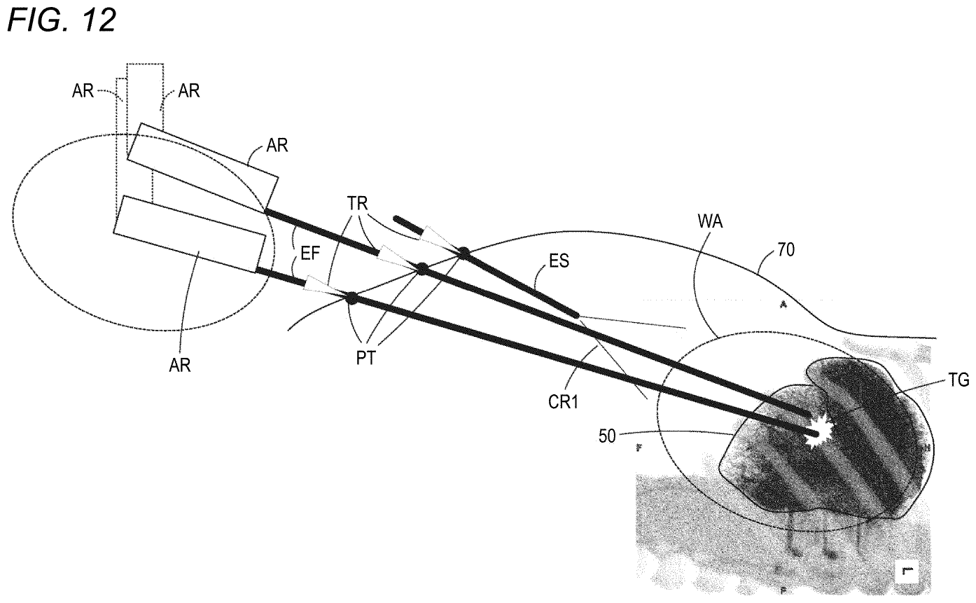

FIG. 12 is a view illustrating an example of a state of the trocar TC, the surgical instrument 30, and the inside of the subject PS during the robotic surgery.

The end effector EF attached to the robot arm AR of the robot main body 320 is inserted into the subject PS through the trocar TC. In FIG. 12, the trocar TC is installed on a body surface 70 of the subject PS to which the pneumoperitoneum is performed. There is also a disease at a part of a liver 50, which is the target TG to be treated. The state near the target TG is imaged by the endoscope ES attached to the robot arm AR During surgery, adjustment is performed such that the vicinity of the target TG is included in the visual field (imaging range CR1) of the endoscope ES. Similar to the end effector EF, the endoscope ES is also inserted into the subject PS through the trocar TC. In the end effector EF and the endoscope ES (surgical instrument 30), the position of the port PT and the position of the trocar TC are the rotation centers. There can be a case where the position of the rotation center of the surgical instrument 30 at the time of surgical planning and the actual port perforation result, is different from each other. In this case, the actual rotation center, which is different from the surgical plan, may be recognized based on the trocar TC reflected in the overview image, for example.

The surgical planning unit 164 plans the working area WA in order to appropriately treat the target TG of the liver 50 with the end effector EF. The working area WA is formed by the position and orientation of each member of the robot main body 320, but as the degree of freedom of the robot arm AR and the end effector EF of the robot main body 320 is high, the working area WA can be set flexibly. For example, by making the position of the arm base AB variable, the robot main body 320 can place the arm base AB that can maximize the benefits of the surplus (degree of freedom higher than 6) degree of freedom of the robot arm AR. Accordingly, it is possible to further suppress interference between the robot arms AR.

The port PT may include a camera port into which the endoscope ES is inserted, an end effector port into which the end effector EF is inserted, an auxiliary port into which the forceps grasped by the assistant are inserted, and the like. There may be the plurality of ports PT for each of the above-described types, and the size of each port PT may be the same or different for each type. For example, the end effector port into which the end effector EF for suppressing organs or the end effector EF with complicated movement in the subject PS is inserted may be larger than the end effector port into which the end effector EF as an electric knife is inserted. The auxiliary port may be planned relatively freely in terms of placement position.

Next, a generation example of the surgical plan by the robotically-assisted surgical device 100 will be described.

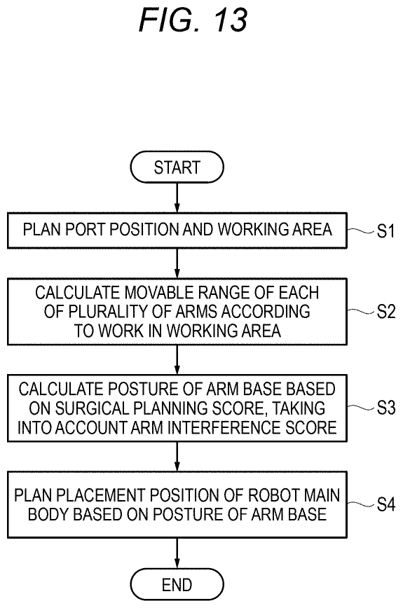

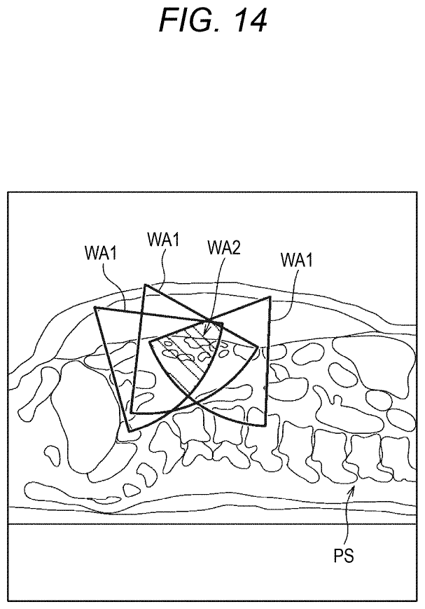

FIG. 13 is a flowchart illustrating a generation example of the surgical plan by the robotically-assisted surgical device 100. The processing illustrated in FIG. 13 is mainly executed by the processing unit 160. FIG. 14 is a view illustrating an example of the working area of the inside of the subject PS.

First, the port position and the working area WA are calculated (planned) (S1). The working area WA corresponds, for example, to an individual working area WA1 or an overall working area WA2 in FIG. 14. A calculation example of the port position and the working area WA will be described later as supplementary information. The calculation method of the port position and the working area WA is not limited to the method illustrated in the supplementary information. For example, the calculation method of the port position and the working area WA illustrated in Reference Literature 1 (U.S. Patent Application Publication 2012/0253515) or Reference Literature 2 (U.S. Patent Application Publication 2014/0148816) may be used.

The movable range of each of the plurality of robot arms AR according to the work (treatment) in the working area WA is calculated (S2). Based on the movable range of each of the plurality of robot arms AR, the arm interference score can be calculated. For example, the smaller the movable range of the robot arm AR, the larger the arm interference score, and this may mean that the greater the interference with other robot arms AR. Meanwhile, the larger the movable range of the robot arm AR, the smaller the arm interference score, and this may mean that the smaller the interference with other robot arms AR.

The posture of the arm base AB of the robot main body 320 is calculated (planned) (S3). For example, the posture of the arm base AB in a case where the surgical planning score that takes into account the arm interference score is equal to or less than a threshold value th5 (for example minimum) is determined as the planned posture of the arm base AB. Accordingly, the robotically-assisted surgical device 100 can plan the posture of the arm base AB with reduced (for example minimized) interference between the plurality of robot arms AR of the robot main body 320.

The placement position of the robot main body 320 is calculated (planned) based on the posture of the arm base AB calculated in S3 (S4). In other words, the placement position of the robot main body 320 in a case of the planned posture of the arm base AB calculated in S3 is calculated. For example, the position of the robot main body 320 in a case where the surgical planning score that takes into account the robot stability score is equal to or greater than a threshold value th6 (for example, maximum) is determined as the planned position of the robot main body 320. Accordingly, the robotically-assisted surgical device 100 can plan the stable placement of the robot main body 320, and can derive the planned position at which the perforation work of the port PT, the equipment of the surgical instrument 30, various treatments and the like are easily performed.

The equipped posture of the robot main body 320 may also be calculated (planned). In other words, the equipped posture of the robot main body 320 in a case of the planned posture of the arm base AB calculated in S3 is calculated. For example, the equipped posture of the robot main body 320 in a case where the surgical planning score is equal to or greater than a threshold value th7 (for example, maximum) is determined as the planned posture related to the equipped posture of the robot main body 320. Accordingly, the robotically-assisted surgical device 100 can optimize the operability of the robot main body 320 when attaching the surgical instrument 30 or when performing various treatments of the robotic surgery.

Next, the operation of the robotically-assisted surgical system 1 during the robotic surgery will be described.

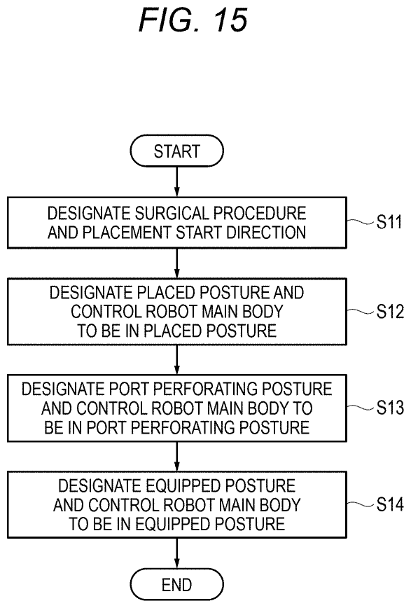

FIG. 15 is a flowchart illustrating an operation example during the robotic surgery by the surgical robot 300. Here, the operations of the assistant or others during the robotic surgery will be described.

First, the processing unit 360 designates the surgical procedure and the placement start direction via the control panel CP before placing the robot main body 320 at the planned position (S11). The placement start direction is information that indicates from which angle (direction) with respect to the subject PS the robot main body 320 is oriented toward the planned position from a position that is away from the subject PS more than a predetermined distance.

The processing unit 360 designates the placed posture as the posture of the robot main body 320 via the control panel CP, and the robot main body 320 is set to the planned posture related to the placed posture (S12). The assistant or others move the robot main body 320 to the planned position in the vicinity of the subject PS, for example, in the placed posture.

The processing unit 360 determines whether or not the robot main body 320 has approached the subject PS. In this case, the processing unit 360 may recognize the distance between the overview camera CA and the subject PS by analyzing the overview image captured by the overview camera CA. In a case where this distance is equal to or less than a threshold value th8, it may be determined that the robot main body 320 has approached the subject PS. The assistant or others confirm the body surface of the subject PS illustrated in the overview image by showing the body surface on the display device 400 and the like, and adjust the position of the robot main body 320 with respect to the subject PS. The assistant or others may perform this position adjustment, for example, based on the planned perforation position of the camera port that was marked before the robot main body 320 started to be placed.

When the robot main body 320 is placed at the planned position, the processing unit 360 designates the port perforating posture as the posture of the robot main body 320 via the control panel CP, and the robot main body 320 is set to the planned posture related to the port perforating posture (S13). The assistant or others perforate the port PT at the planned position of the port in a state where the robot main body 320 is in the port perforating posture.

When the port PT is perforated, the processing unit 360 designates the equipped posture as the posture of the robot main body 320 via the control panel CP, and the posture of the robot main body 320 is set to the planned posture related to the equipped posture (S14). Accordingly, the posture of the robot main body 320 (especially, the arm base AB and each robot arm AR) is set to a posture in which the surgical instrument 30 can be easily equipped.

The assistant or others install the endoscope ES on the camera arm and insert the endoscope ES into the subject PS via the trocar TC installed in the camera port. The position (three-dimensional position) of the camera port is the position of the trocar TC and is the rotation center of the endoscope ES. The robot main body 320 may determine this rotation center, for example, based on the surgical plan (for example, kinematics of the robot main body 320) and the overview image. The assistant or others may move the endoscope ES via the robot operation terminal 310 and search for the rotation center of other surgical instruments 30 attached to the camera arm. In this case, the processing unit 360 of the robot main body 320 may input and determine the rotation center of the other surgical instruments 30 via the control panel CP. The surgical instrument 30 for determining the rotation center may be a dedicated surgical instrument for determining the rotation center. Accordingly, the robot main body 320 can adjust the position of the rotation center even when, for example, the planned position of the port PT and the position of the actually perforated port PT are shifted from each other and the rotation center is shifted from the planned position, and can suppress a decrease in the accuracy of robotic surgery.

Similar to endoscope ES, the assistant or others install the end effector EF on the end effector arm, and insert the end effector EF into the subject PS via the trocar TC installed in the end effector port. The robot main body 320 may also determine the rotation center of the end effector EF using the same method as that for the endoscope ES.

In this manner, during the robotic surgery, the port PT is not perforated before the robot main body 320 is placed, but the port PT (especially, the end effector port) is perforated after the robot main body 320 is placed.

In the robot main body of the surgical robot in the related art, the surgical instrument 30 is slidable along the robot arm AR in the slidable range, but the movement of the robot arm AR is restricted since the robot arm AR is connected to the trocar TC. Meanwhile, in the robot main body 320, the trocar TC, into which the surgical instrument 30 attached to the robot arm AR is inserted, is not connected to the robot arm AR, and the robot arm AR and the surgical instrument 30 are connected to each other. Accordingly, the surgical instrument 30 can be slid by the slide mechanism, and the surgical instrument 30 can be moved back and forth in the axial direction of the surgical instrument 30 by the movement of the robot arm AR. Therefore, the degree of freedom in positioning the robot arm AR increases. According to this, the robot main body 320 can reduce the interference of the robot arm AR.

Next, the movement of the robot main body 320 when being placed at the planned position will be described.

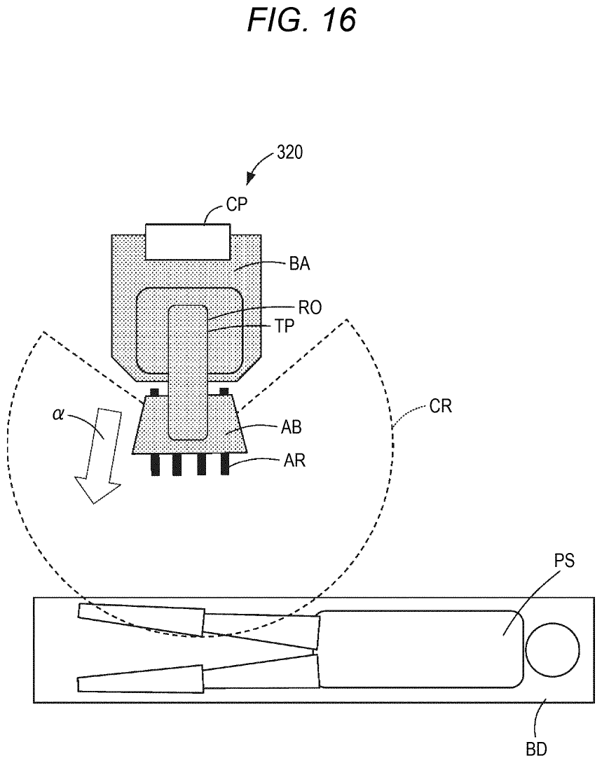

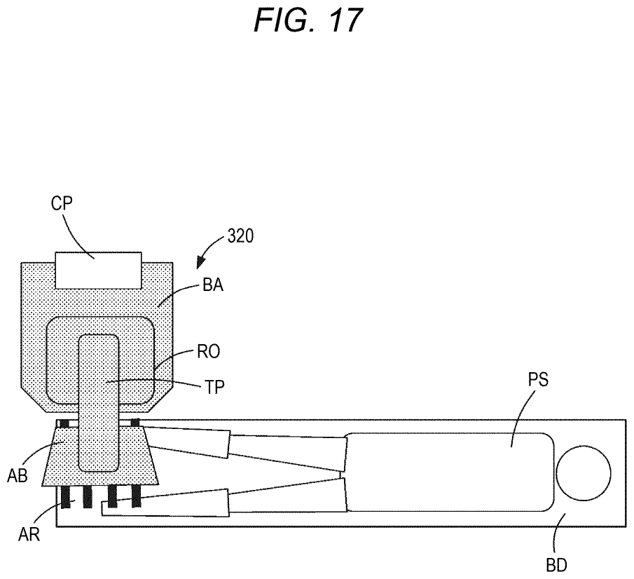

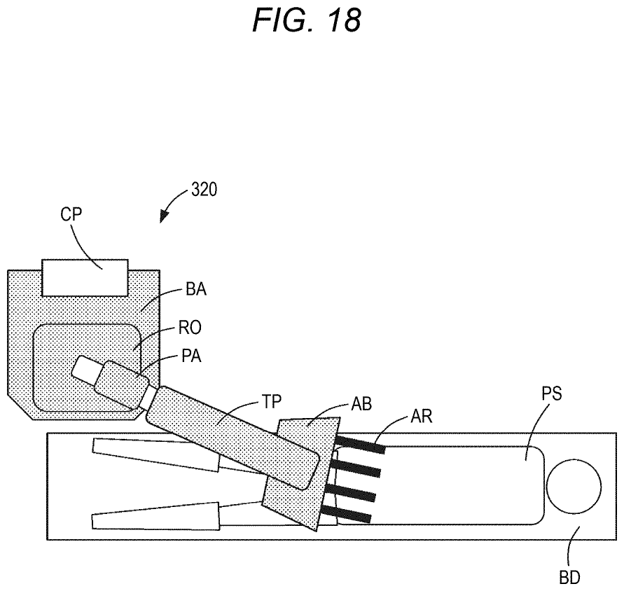

FIG. 16 is a schematic view illustrating an approaching example of the robot main body 320 to the subject PS. FIG. 17 is a schematic view illustrating a placement example of the robot main body 320 at a planned position in the vicinity of the subject PS. FIG. 18 is a schematic view illustrating an example of the robot main body 320 in the port perforating posture at the planned position of the robot main body 320. FIGS. 16 to 18 all illustrate a state of the vicinity of the subject PS placed on the surgical bed BD in the operating room, viewed from the ceiling side of the operating room.

The robot main body 320 enters the operating room, advances in a direction of arrow α, and approaches the subject PS placed on the surgical bed BD (refer to FIG. 16). While approaching the subject PS, the overview camera CA captures the subject included in an imaging range CR of the overview camera CA. The imaging range CR includes at least a part of the subject PS. The robot main body 320 is in a placed posture while approaching the subject PS. The state of the surrounding of the subject PS is also reflected in the imaging range CR. Therefore, for example, by displaying an overview image on the control panel CP or the display device 400, the assistant or others can move the robot main body 320 to the planned position while paying attention to the surrounding of the robot main body 320.

The robot main body 320 is placed at the planned position in the vicinity of the subject PS, for example (refer to FIG. 17). The robot main body 320 takes the port perforating posture after being placed at the planned position (refer to FIG. 18). The port PT is perforated into the subject PS even before the robot main body 320 is placed at the planned position, and the posture of the robot main body 320 may be shifted from the placed posture to the equipped posture without shifting from the placed posture to the port perforating posture.

Next, the assistance in the port registration by the robotically-assisted surgical system 1 will be described.

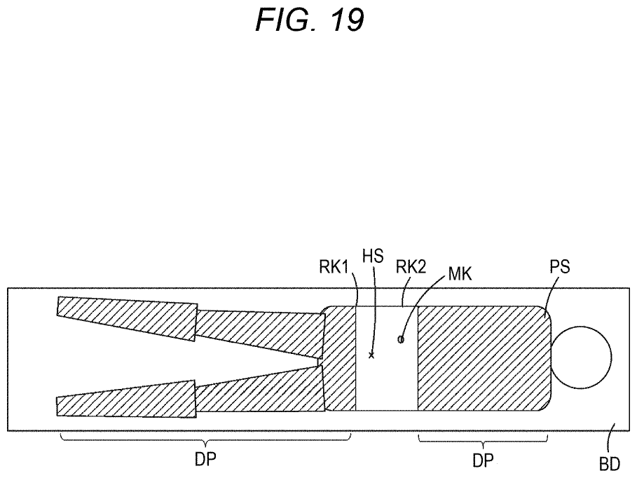

FIG. 19 is a schematic view illustrating an example of a landmark of the subject PS. For example, in a case where the overview image includes the landmark of the subject PS, the processing unit 360 of the robot main body 320 can use the landmark as reference position for the registration in the subject PS based on the results of image analysis of the overview image.

FIG. 19 illustrates that the neighborhood of the abdomen of the subject PS is the surgery target. The neighborhood of the abdomen, which is the surgery target, is not covered with a drape DP, and the part other than the neighborhood of the abdomen, which is the surgery target, is covered with the drape DP. There is no landmark at the part covered with the drape DP in the subject PS. At the part that is not covered with the drape DP in the subject PS, the body surface of the subject PS can be visually recognized. There may be no drape DP.

The landmark of the subject PS may include an umbilical HS, a contour RK1 of pelvis, a contour RK2 of body, other markings MK drawn on the subject PS, and the like. The landmarks may also include axillary lines, sword-shaped projections, a contour of rib, and the like.

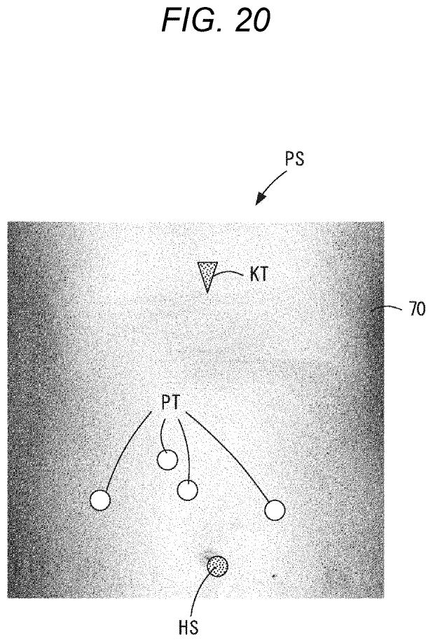

FIG. 20 is a schematic view illustrating a display example in which the landmark of the subject PS and the planned position of the port PT are superimposed on the three-dimensional image of the body surface of the subject PS. FIG. 20 illustrates, for example, an example of a state of the body surface 70 of the subject PS during a surgical simulation before surgery. In FIG. 20, a sword-shaped projection KT (for example, distal end of the sword-shaped projection KT) and the umbilical HS as landmarks and the planned position of the port PT are illustrated at the corresponding positions on the body surface 70 of the subject PS. The image (the image in FIG. 20) in which the information indicating the landmark and the planned position of the port PT are superimposed on the body surface image of the subject PS may be displayed on the control panel CP or the display device 400. The information indicating the landmarks may not be superimposed and displayed.

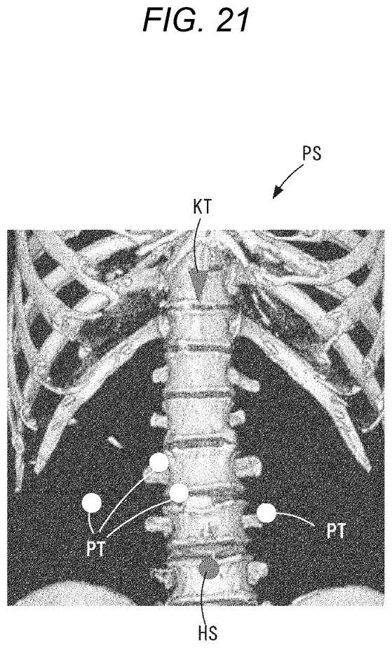

FIG. 21 is a schematic view illustrating a display example in which the landmark of the subject PS and the planned position of the port PT are superimposed on the three-dimensional image (for example, raycast image) of the inside of the body of the subject PS. FIG. 21 illustrates, for example, an example of a state of the inside of the body of the subject PS during the surgical simulation before surgery. In FIG. 21, the sword-shaped projection KT (for example, distal end of the sword-shaped projection KT) and the umbilical HS as landmarks and the planned position of the port PT are illustrated at the corresponding positions on the three-dimensional image of the subject PS. The image (the image in FIG. 21) in which the information indicating the landmark and the planned position of the port PT are superimposed on the three-dimensional image of the subject PS may be displayed on the control panel CP or the display device 400. The information indicating the landmarks may not be superimposed and shown.

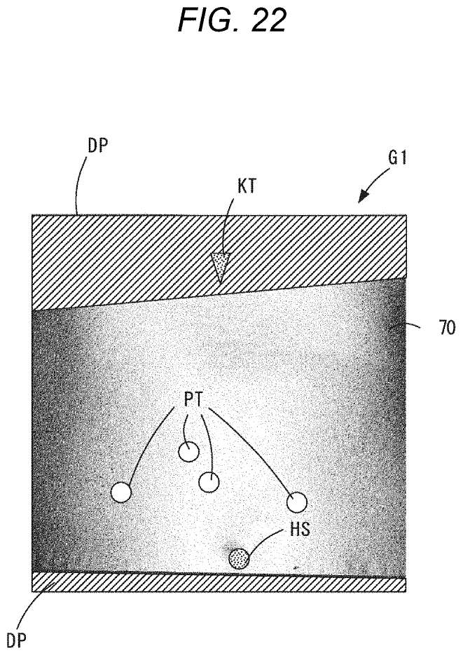

FIG. 22 is a schematic view illustrating an example of the landmark of the subject PS included in the overview image G1 and the planned position of the port PT. FIG. 22 illustrates, for example, an example of the state of the body surface 70 reflected in the overview image G1 captured by the overview camera CA during surgery. In FIG. 22, a part of the body surface 70 is covered with the drape DP. In FIG. 22, the sword-shaped projection KT (for example, distal end of the sword-shaped projection KT) and the umbilical HS as landmarks and the planned position of the port PT are illustrated at the corresponding positions on the overview image G1. The image (the image in FIG. 22) in which the information indicating the landmark and the planned position of the port PT is superimposed on the overview image G1 may be shown on the control panel CP or the display device 400. The information indicating the landmarks may not be superimposed and shown. The overview image G1 may be used, for example, as a real-time image during the port perforation work, and may be superimposed and shown with the planned position of the port.

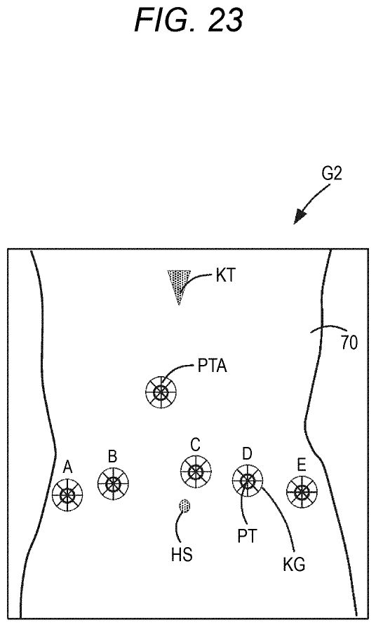

FIG. 23 is a schematic view illustrating an example of the landmark of the subject PS included in the overview image, the planned position of the port PT, and tolerance information KG of the port PT. FIG. 23 illustrates, for example, an example of the state of the body surface 70 reflected in an overview image G2 captured by the overview camera CA during surgery. In FIG. 23, the body surface 70 is not covered with the drape DP. In FIG. 23, the sword-shaped projection KT (for example, distal end of the sword-shaped projection KT) and the umbilical HS as landmarks, the planned position of the port PT, and the tolerance information KG of the port PT are illustrated at the corresponding positions on the overview image G2. The image (the image in FIG. 23) in which the information indicating the landmark, the planned position of the port PT, and the tolerance of the port PT is superimposed on the overview image G2 may be shown on the control panel CP or the display device 400. The information indicating the landmarks may not be superimposed and shown.

In FIG. 23, the plurality of ports PT are illustrated. The plurality of ports PT include an auxiliary port PTA, the camera port, the end effector port, and the like. Around the planned position of each port PT (PTA and A to E), the tolerance range corresponding to the tolerance information KG of the port PT is illustrated. In FIG. 23, the shape of the outer circumference of the tolerance range is circular as an example, but may be any other shape.

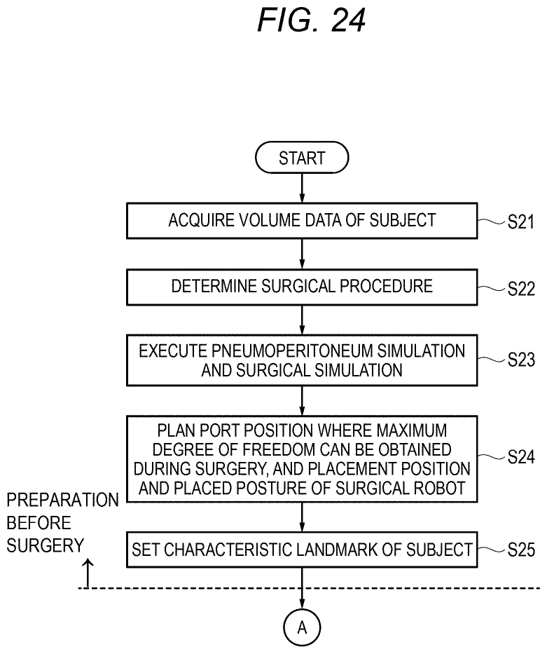

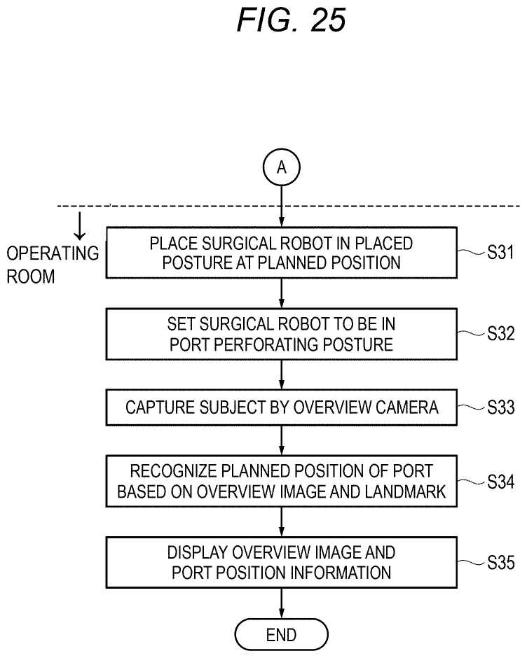

FIGS. 24 and 25 are flowcharts illustrating an operation example related to the port registration by the robotically-assisted surgical system 1. S21 to S25 in FIG. 24 are performed, for example, before surgery. Each processing here is performed, for example, by each unit of the processing unit 160 of the robotically-assisted surgical device 100. S31 to S35 in FIG. 25 are performed, for example, during surgery (for example, before the actual treatment). Each processing here is performed, for example, by the processing unit 360 of the robot main body 320.

Before surgery, the processing unit 160 acquires the volume data of the subject PS (for example, a patient) (S21). For example, the surgical procedure is designated (for example, selected) via the UI 120 (S22). The pneumoperitoneum simulation and the surgical simulation are performed (S23). The port position and the placement position of the robot main body 320 are planned based on the surgical planning score that takes into account, for example, at least one of the port position score, the arm interference score, and the robot stability score (S24). Each posture (for example, the placed posture, the port perforating posture, and the equipped posture) of the robot is planned (S24) based on the above-described method, for example. For example, by taking into account the arm interference score, the port position where the maximum degree of freedom can be obtained during surgery, and the planned position and the planned posture of the robot main body 320, can be obtained.

The landmark of the subject PS is set (S25). For example, the position of the landmark may be designated and set for the volume data of the subject PS via the UI 120. The number of landmarks to be set may be one or more. The planned position of the port PT, the planned position of the robot main body 320, the planned posture of the robot main body 320, the setting information on landmark, and the like are included in the surgical plan, the surgical plan is stored in the memory 150, and the surgical plan is transmitted to the robot main body 320 via the transmission/reception unit 110 (S25). The robot main body 320 receives the surgical plan via the transmission/reception unit 321 and holds the surgical plan in the memory MR.

During surgery, the processing unit 360 acquires the surgical plan from the memory MR. The planned posture related to the placed posture and the planned position of the robot main body 320 are acquired from the surgical plan. The robot main body 320 is controlled to take the placed posture based on the planned posture (S31). In a state of the placed posture, the assistant or others move the robot main body 320 to the planned position. In this case, the processing unit 360 may display the predetermined guidance information for moving the robot main body 320 to the planned position on the control panel CP or the display device 400, and the assistant or others may move the robot main body 320 after confirming the display of the guidance information.

The planned posture related to the port perforating posture is acquired from the surgical plan. The robot main body 320 is controlled to take the port perforating posture based on the planned posture (S32).

The overview camera CA captures the subject PS (S33). The processing unit 36A) acquires the overview image that includes at least a part of the subject imaged by the overview camera CA. The overview image may be captured before the robot main body 320 enters the operating room, or may be captured after the robot main body 320 enters the operating room. The overview image may be captured in a case where the distance between the robot main body 320 and the subject PS is equal to or less than a threshold value th9, that is, after the robot main body 320 approaches the subject PS. The overview image may be captured after the robot main body 320 is placed at the planned position. The overview image may be captured continuously during the robotic surgery.

Based on the overview image and the landmarks of the subject PS, the planned position of each port PT in the overview image is recognized (S34).

As a specific example of S34, information on the setting of landmarks and the information on the planned position of each port PT with respect to the landmarks of the subject PS are acquired from the surgical plan. The overview image is analyzed to recognize the position (image position) of the landmarks of the subject PS. The planned position (image position) of each port PT in the overview image is recognized based on the image position of the landmark set in the overview image and the planned position of each port PT with respect to the landmark.

The port position information that indicates the planned position of each port PT recognized in the overview image, is superimposed on the overview image and shown on the control panel CP or the display device 400 (S35). In this case, the tolerance information of the port PT may be displayed together with the port position information.

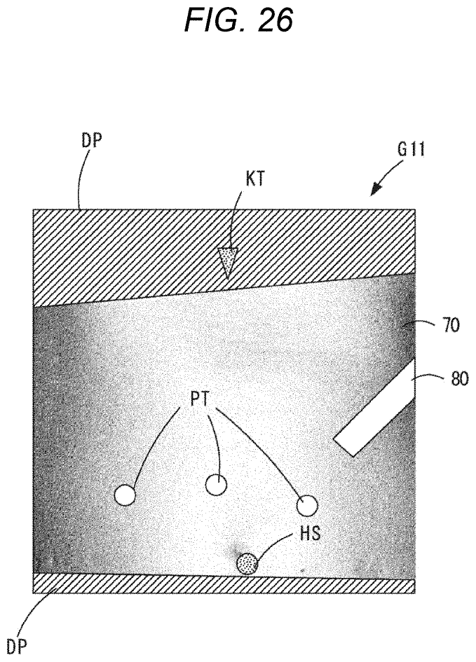

By confirming the port position information and the tolerance information displayed on the control panel CP or the display device 400, the assistant or others can recognize the planned position of the port PT on the body surface 70 of the subject PS in actual space and the tolerance thereof. When the assistant or others actually perforate the port PT, a perforating instrument 80 for perforating the port PT is reflected in an overview image G11 (refer to FIG. 26). Therefore, the assistant or others can visually confirm the positional relationship between the planned position of the port PT in the overview image G11 and the perforating instrument 80, and can perforate the port PT with high accuracy.

In this manner, the robotically-assisted surgical system 1 can display the port position information and the tolerance information using the overview image by performing the operations related to the port registration. Accordingly, the assistant or others who have confirmed this display can guide the position of the port to be perforated to the planned position of the port PT indicated by the port position information, and can assist in perforating the port PT.

By setting landmarks, the robotically-assisted surgical system 1 can register the coordinate system (coordinate system of the subject) of the 3D data such as the volume data and the models with respect to the landmarks, with the coordinate system (coordinate system of the robot) during surgery captured by the overview camera CA placed on the robot main body 320. Accordingly, the robotically-assisted surgical system 1 can superimpose and shown the planned position of the port on the overview image. The registration may be performed any number of times, may be performed after the robot main body 320 is placed at the planned position, or may be performed after entering the operating room and before the robot main body 320 is placed at the planned position.

Next, the variations of the embodiment will be described.

This embodiment illustrates that the information on the intraoperative navigation (for example, the port position information and the tolerance information) is displayed on the control panel CP or the display device 400, but the invention is not limited thereto. For example, the arm base AB may include a projector, and the information on the intraoperative navigation may be displayed by being projected onto the body surface of the subject PS. For example, the robot main body 320 may include a speaker, or a speaker may be installed in the operating room. The speaker may output audio information indicating the planned position of the port PT in the subject PS and the tolerance range. Other presentation methods may be used to present information on the intraoperative navigation.

In a case where the overview image is continued to be captured by the overview camera CA for a predetermined period of time including the time of the port registration, the port PT perforated by the assistant or others may be reflected in the overview image. The processing unit 360 may perform image analysis on the overview image and recognize the perforated port PT The information on the recognized port PT may be used for various treatments in robotic surgery. For example, the processing unit 160 of the robotically-assisted surgical device 100 may acquire information on the perforation position (the actual perforated position) of the port PT in the subject PS via the transmission/reception unit 110. The processing unit 160 may update the port position in the 3D data recognized by the robotically-assisted surgical device 100 from the planned position of the port to the port perforation position. Accordingly, the robotically-assisted surgical device 100 registers the port position of the 3D data in the virtual space with the actual port position in the actual space, and to perform navigation using the 3D data with even higher accuracy.

The processing unit 360 may generate the guidance information for guiding the perforating instrument 80 to the planned position of the port PT based on the position of the perforating instrument 80 reflected in the overview image and the planned position of the port PT. The processing unit 360 may display this guidance information on the control panel CP or the display device 400.

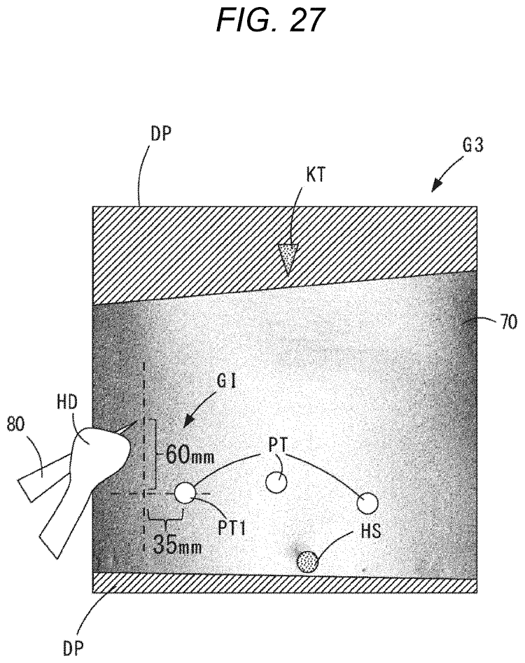

FIG. 27 is a view illustrating a display example of guidance information G1 for guiding the perforating instrument 80 to the planned position of the port PT.

An overview image G3 in FIG. 27 illustrates the position information of three ports. It is assumed that the assistant or others perforate a port PT1 among the planned positions of the three ports PT with the perforating instrument 80. In the overview image G3, a hand HD of the assistant or others and a part of the perforating instrument 80 grasped by the hand HD are reflected.

The processing unit 360 analyzes the overview image G3 and recognizes the position of the perforating instrument 80 in the overview image G3. The processing unit 360 recognizes the planned position of the port PT1 in the overview image G3. The processing unit 360 may calculate the difference between the recognized position of the perforating instrument 80 and the planned position of the port PT, and generate the guidance information G1 based on this difference. In this case, the moving direction from the position of the perforating instrument to the planned position of the port PT1 and the distance (moving distance) between the position of the perforating instrument 80 and the planned position of the port PT, may be calculated. Accordingly, the guidance information including the above-described moving direction and the moving distance may be shown.

In FIG. 27, the guidance information G1 indicates that the port PT1 can be reached by moving 60 mm from the perforating instrument 80 in the downward direction in the drawing (a direction toward the foot along the body axis direction of the subject PS) and by moving 35 mm from the position in the rightward direction in the drawing.

By confirming the guidance information G1 displayed together with the overview image G3, the assistant or others can intuitively determine in which direction and to what extent the perforating instrument 80 should be moved. The guidance information G1 may be shown in a state other than the state illustrated in FIG. 27. For example, the guidance information G1 may be displayed as arrows. In this case, the direction of the arrow may indicate the moving direction, and the size and length of the arrow may indicate the moving distance.

Furthermore, the processing unit 360 may show the following information on the control panel CP or the display device 400 as information to assist the assistant or others in the perforation of the port PT.

For example, the processing unit 360 may show the guidance information for moving the perforating instrument 80 to the specific port PT among the plurality of ports PT. In this case, the specific port PT may be designated via the control panel CP. The specific port PT may be the port PT closest to the perforating instrument 80 recognized in the overview image. The specific port PT can be one or more. The processing unit 360 may show the above-described guidance information for each of all of the plurality of ports PT.

The processing unit 360 may also show the port position information depending on whether or not the port PT has been perforated by the assistant or others. The processing unit 360 may determine whether or not each port PT has been perforated, for example, based on image analysis of the overview image. The processing unit 360 may acquire designation information for designating the port PT that has been perforated, via the control panel CP, and determine that the designated port PT has been perforated. The processing unit 360 can show the port position information of the port PT that has not been perforated and complete the display of the port position information of the port PT that has been perforated. The processing unit 360 may change the display mode of the port position information on the port PT that has been perforated and may be different from the display mode of the port position information on the port PT that has not been perforated. The processing unit 360 may also show the guidance information for guiding the user to all or a part of the ports PT that have not been perforated.

The actuator AC of the robot main body 320 may also have a function of driving the tires and the like of the robot main body 320. Accordingly, the robot main body 320 can move automatically to the planned position. In this case, the processing unit 360 may set the automatic operation mode as the operation mode of the robot main body 320 based on the operation of the control panel CP. The operation mode may include, for example, a manual operation mode in which the assistant or others push and move the robot main body 320, and an automatic operation mode in which the robot main body 320 moves by automatic operation.

The processing unit 360 may analyze the overview image G3 to recognize marks made on the body surface of the subject PS by the assistant or others. Accordingly, the assistant or others can draw additional landmarks on the body surface of the subject PS. The addition of landmarks may be planned in advance in the preoperative planning. The added landmarks may be marked for the purpose of clarifying obscure landmarks in the overview image. This landmark may include, for example, a line traced between the sternum. The preoperative plan may be included in the surgical plan.

In the preoperative planning, the surgical planning unit 164 may use prepared 3D model data to plan the port position instead of the 3D data of the subject PS. The port positions included in the prepared 3D model data may be used for planning the port positions. The 3D model data may be customized by the model setting unit 163 according to the characteristics of the subject PS. According to this, the robotically-assisted surgical device 100 can reduce the effort required for the preoperative planning in typical or simple cases.

In the preoperative planning, the surgical planning unit 164 may use 2D data of the subject PS or prepared 2D model data to plan the port position instead of the 3D data of the subject PS. According to this, the robotically-assisted surgical device 100 can reduce the effort required for the preoperative planning in typical or simple cases.

[Supplement to Surgical Simulation and Surgical Plan]

Next, the surgical plan by the robotically-assisted surgical device 100 will be supplemented. First, a calculation example of the port position score will be described.

The plurality of port positions may be defined according to, for example, the surgical procedure, and may be assumed to be placed respectively at any position on the body surface of the subject PS. Accordingly, various combinations of port positions may be assumed as well as combinations of the plurality of port positions. From one port PT, one end effector EF attached to the robot arm AR can be inserted into the subject PS. Accordingly, from the plurality of ports PT, the plurality of end effectors EF attached to the plurality of robot arms AR can be inserted into the subject PS.

The range that can be reached by one end effector EF in the subject PS through the port PT is the working area (individual working area WA1) (refer to FIG. 14) where the work (treatment in the robotic surgery) is possible by one end effector EF. Accordingly, the area where the individual working areas WA1 by plurality of end effectors EF overlap becomes a working area (overall working area WA2) that the plurality of end effectors EF can reach simultaneously in the subject PS via the plurality of ports PT (refer to FIG. 14). Since the treatment according to the surgical procedure requires a predetermined number (for example three) of end effectors EF to work simultaneously, the overall working area WA2 that can be reached by the predetermined number of end effectors EF simultaneously is considered.

The position in the subject PS where the end effector EF can reach varies depending on the kinematics of the robot main body 320, and thus, this fact is taken into account in deriving the port position which is the position where the end effector EF is inserted into the subject PS. The position of the overall working area WA2 in the subject PS to be ensured differs depending on the surgical procedure, and thus, this fact is taken into account in deriving the port position corresponding to the position of the overall working area WA2.

The surgical planning unit 164 may calculate the port position score for each combination of the plurality of acquired (assumed) port positions. The surgical planning unit 164 may plan a combination of port positions that becomes the port position score (for example, a port score that is the maximum) that satisfies a predetermined condition, among the assumed combinations of the plurality of port positions. In other words, the plurality of port positions included in the combination of port positions may be used as the planned positions of the plurality of ports which are perforation targets.

The relationship between the port position and the operation of the movable unit of the surgical robot 300 may satisfy the relationship described in, for example, Reference Non-patent Literature 2 (Mitsuhiro Hayashibe, Naoki Suzuki, Makoto Hashizume, Kozo Konishi, Asaki Hattori, “Robotic surgery setup simulation with the integration of inverse-kinematics computation and medical imaging”, computer methods and programs in biomedicine, 2006, P63-P72) and Reference Non-patent Literature 3 (Pal Johan From, “On the Kinematics of Robotic-assisted Minimally Invasive Surgery”, Modeling Identication and Control, Vol. 34, No. 2, 2013, P69-P82).

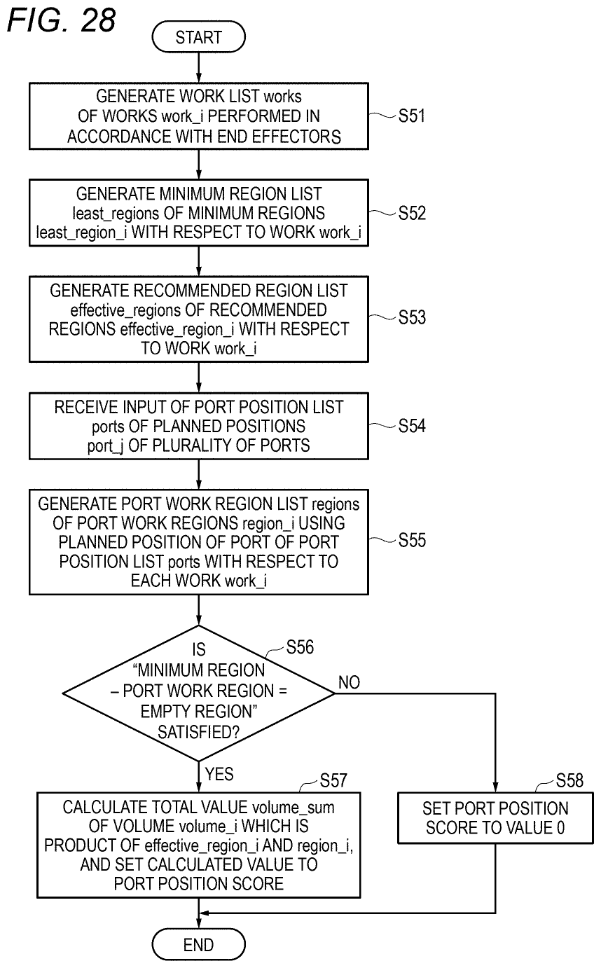

FIG. 28 is a flowchart illustrating an operation example of a case of calculating the port position score by the robotically-assisted surgical device 100. The initial value of the port position score is 0. The port position score is an evaluation function (evaluation value) that indicates the value of a combination of port positions. A variable i is an example of work identification information, and a variable j is an example of port identification information.

The surgical planning unit 164 generates a work list works, which is a list of works work_i using each end effector EF, according to the surgical procedure (S51). The work work_i contains information for each end effector EF to work in the order of surgery according to the surgical procedure. The work work_i may include, for example, grasping, excision, suturing, and the like. The work may include independent work by a single end effector EF or cooperative work by a plurality of end effectors EF.

The surgical planning unit 164 determines a minimum region least_region_i, which is the minimum region required to perform the work work_i contained in the work list works, based on the surgical procedure and the volume data of the virtual pneumoperitoneum state (S52). The minimum region may be defined by the three-dimensional region in the subject PS. The surgical planning unit 164 generates a minimum region list least-regions, which is a list of minimum regions least_region_i (S52).

The surgical planning unit 164 determines a recommended region effective_region_i, which is a region recommended for performing the work work_i contained in the work list works, based on the surgical procedure, the kinematics of the robot main body 320, and the volume data of the virtual pneumoperitoneum state (S53). The surgical planning unit 164 generates recommended region list effective_regions, which is a list of recommended regions effective_regions_i (S53). The recommended region may include, for example, the recommended space for the end effector EF to operate, along with the minimum space for performing work (minimum region).

The surgical planning unit 164 acquires information on the port position list ports, which is a list of the plurality of port positions port_j (S54). The port position may be defined in three-dimensional coordinates (x, y, z). The surgical planning unit 164 may, for example, receive user input via the UI 120 and acquire a port position list ports that include one or more port positions designated by the user. The surgical planning unit 164 may acquire the port position list ports stored as a template in the memory 150.

The surgical planning unit 164 determines the port work region region_i, which is a region in which each end effector EF can work through each port position port_j for each work work_i, based on the surgical procedure, the kinematics of the robot main body 320, the volume data of the virtual pneumoperitoneum state, and the plurality of acquired port positions (S55). The port work region may be defined as a three-dimensional region. The surgical planning unit 164 generates port work region list regions, which is a list of port work region region_i (S55).

The surgical planning unit 164 calculates a subtraction region (subtraction value) by subtracting the port work region region_i from the minimum region least_region_i for each work work_i (S56). The surgical planning unit 164 determines whether or not the subtraction region is an empty region (the subtraction value is a negative value) (S56). Whether or not the subtraction region is an empty region indicates whether or not there is a region (a region that is not reached by the end effector EF through the port PT) that is not covered with the port work region region_i, at least at a part within the minimum region least_region_i.

In a case where the subtraction region is an empty region, the surgical planning unit 164 calculates a volume value volume_i, which is the product of the recommended region effective_region_i and the port work region region_i (S57). The surgical planning unit 164 then sums the volume value volume_i calculated for each work work_i and calculates a total value volume_sum. The surgical planning unit 164 sets the total value volume_sum to the port position score (S57).

In other words, in a case where the subtraction region is an empty region, there is no region which is not covered with the port work region within the minimum region, it is preferable that this port position list ports (combination of port positions port_j) is selected, and thus, the value for each work work_i is added to the port position score such that the port position list is easily selected. By determining the port position score based on the volume volume_i, the larger the minimum region or the port work region, the larger the port position score, and the easier it is to select the port position list ports. Accordingly, the surgical planning unit 164 can easily select a combination of port positions that have a large minimum region or the port work region and that make each treatment easy in surgery.

Meanwhile, in a case where the subtraction region is not an empty region, the surgical planning unit 164 sets the port position score for the port position list ports to the value 0 (S58). In other words, there is a region which is not covered with the port work region at least at a part within the minimum region, there is a possibility that the work of target work work_i cannot be completed, and thus, it is not preferable that this port position list ports are selected. Therefore, the surgical planning unit 164 sets the port position score to the value 0 and excludes this port position list from the selection candidates such that this port position list ports are less likely to be selected. In this case, the surgical planning unit 164 sets the overall port position score to the value 0, even when the region is an empty region in a case where other work work_i is performed using the same port position list ports.

The surgical planning unit 164 may repeat each step of FIG. 28 for all work work_i and calculate the port position score taking all work work_i into account.

In this manner, by deriving the port position score, the robotically-assisted surgical device 100 can grasp how appropriate the combination of port positions for perforation candidates is, in a case where the robotic surgery is performed using the plurality of port positions provided on the body surface of the subject PS. The individual working area WA1 and the overall working area WA2 depend on the placement positions of the plurality of ports which are perforation targets. Even in this case, by taking into account the score (port position score) for each combination of the plurality of port positions, the surgical robot 300 can derive a combination of the plurality of port positions in which, for example, the port position score is equal to or greater than a threshold value th10 (for example, the maximum), and can set the combination as the planned position of the port where the robotic surgery is easily performed.

By appropriately ensuring the working area WA based on the port position score, the user can ensure a wider visual field in the subject PS, which cannot be seen directly in robotic surgery, can ensure a wider port work region, and can easily deal with an unexpected situation.

In robotic surgery, the perforated port position is unchanging, but the robot arm AR to which the end effector EF to be inserted into the port position is attached can move within a predetermined range. Therefore, in robotic surgery, planning the port position is important because the robot arms AR can interfere with each other depending on the planned position of the port. Since the positional relationship between the surgical robot 300 and the subject PS is not changed during surgery in principle, it is important to plan the port position.

Next, the details of port position adjustment will be described.

The surgical planning unit 164 acquires information on the plurality of port positions (candidate positions) based on, for example, templates stored in the memory 150 or user instructions via the UI 120. The surgical planning unit 164 calculates the port position score in a case of using this plurality of port positions based on the combination of the acquired plurality of port positions.

The surgical planning unit 164 may adjust the position of the port PT based on the port position score. In this case, the surgical planning unit 164 may adjust the position of the port PT based on the acquired port position score in a case of the plurality of port positions and the port position score in a case where at least one of the plurality of port positions is changed. In this case, the surgical planning unit 164 may take into account the minute movement or differentiation of the port position along each direction (x-direction, y-direction, and z-directions) in the three-dimensional space.

For example, the surgical planning unit 164 may calculate a port position score F (ports) for the plurality of port positions according to (Expression 1), and calculate a differential value F′ of F.

F(port_j(x+Δx,y,z))−F(port_j(x,y,z))

F(port_j(x,y+Δy,z))−F(port_j(x,y,z))

F(port_j(x,y,z+Δz))−F((port_j(x,y,z)) (Expression 1)

In other words, the surgical planning unit 164 calculates the port position score F in a case of the port position F(port_j(x+Δx, y, z)), calculates the port position score F in a case of the port position F(port_j(x, y, z)), and calculates the difference therebetween. This difference value indicates the change in the port position score F with respect to a minute change in the x-direction at the port position F(port_j(x, y, z)), that is, the differential value F′ of F in the x-direction.

In other words, the surgical planning unit 164 calculates the port position score F in a case of the port position F(port_j(x, y+Δy, z)), calculates the port position score F in a case of the port position F(port_j(x, y, z)), and calculates the difference therebetween. This difference value indicates the change in the port position score F with respect to a minute change in they-direction at the port position F(port_j(x, y, z)), that is, the differential value F′ of F in the y-direction.

In other words, the surgical planning unit 164 calculates the port position score F in a case of the port position F(port_j(x, y, z+Δz)), calculates the port position score F in a case of the port position F(port_j(x, y, z)), and calculates the difference therebetween. This difference value indicates the change in the port position score F with respect to a minute change in the z-direction at the port position F(port_j(x, y, z)), that is, the differential value F′ of F in the z-direction.

The surgical planning unit 164 calculates the maximum value of the port position score based on the differential value F′ in each direction. In this case, the surgical planning unit 164 may calculate the port position with the maximum port position score according to the re-descent method, based on the differential value F′. The surgical planning unit 164 may adjust the port position and optimize the port position such that the calculated port position is the planned position of the port. The planned position of the port may not be the port position with the maximum port position score, for example, may be the position where the port position score is equal to or greater than a threshold value th11, and the port position score may be improved (become higher).

The surgical planning unit 164 may apply the adjustment of the port position to the adjustment of other port positions included in the combination of the plurality of port positions, or to the adjustment of the port position in other combinations of the plurality of port positions. Accordingly, the surgical planning unit 164 can plan the plurality of ports PT with each port position adjusted (for example, optimized) to the port position which is the perforation target.

The plurality of port positions (coordinates of the port positions) can have an error of approximately a predetermined length (for example, 25 mm) between the planned perforation position and the actual perforation position, and the planned accuracy of the port positions is considered to be sufficient at most 3 mm. Therefore, the surgical planning unit 164 may make the plurality of port positions included in a combination of port positions as the planned perforation positions in a brute-force manner for each predetermined length on the body surface of the subject PS, and calculate the port position scores for each of the plurality of port positions. In other words, the planned perforation positions may be placed in a grid pattern of a predetermined length (for example, 3 mm) on the body surface of the subject PS. In a case where the number of ports assumed on the body surface (for example, the number of intersection points in a grid pattern) is n and the number of ports included in the combination of port positions is in, the surgical planning unit 164 may select and combine m port positions from n port positions in order, and may calculate the port position score in each of the combinations. In this manner, in a case where the grid is not excessively fine similar to a grid pattern with 3 mm intervals, the excessive computational load of the surgical planning unit 164 can be suppressed, and the port position scores for all combinations can be calculated.

The surgical planning unit 164 may adjust the plurality of port positions according to known methods. The surgical planning unit 164 may set the planned positions of the port positions as a plurality of port positions included in the combination of the adjusted port positions. Known methods of port position adjustment may include the techniques described in the following Reference Non-patent Literature 4 (Shaun Selha, Pierre Dupont, Robert Howe, David Torchiana. “Dexterity optimization by port placement in robot-assisted minimally invasive surgery”, SPIE International Symposium on Intelligent Systems and Advanced Manufacturing, Newton, Mass., 28-31, 2001), Reference Non-patent Literature 5 (Zhi Li, Dejan Milutinovic, Jacob Rosen, “Design of a Multi-Arm Surgical Robotic System for Dexterous Manipulation”, Journal of Mechanisms and Robotics, 2016), and Reference Non-patent Literature 3 (U.S. Patent Application Publication 2007/0249911)

Similar to the above-described port position adjustment, the surgical planning unit 164 may adjust the equipped posture (including the posture during each treatment during surgery) of the robot main body 320. For example, the equipped posture may be adjusted based on the surgical planning score. In this case, the surgical planning unit 164 may adjust the equipped posture based on the surgical planning score in a case of the equipped posture and the surgical planning score in a case where this equipped posture is changed. In this case, the surgical planning unit 164 may take into account the minute movement or differentiation of the planned position of the robot main body 320 along each direction (x-direction and y-direction) in the two-dimensional plane. The surgical planning unit 164 may also take into account minute movement or differentiation of the posture of the arm base AB in the three-dimensional space.

For example, the surgical planning unit 164 may calculate a surgical planning score FA(β) with respect to a posture β of the arm base AB in the equipped posture according to (Expression 1A), and calculate a differential value FA′ of FA.

The surgical planning unit 164 calculates the maximum value of the surgical planning score based on the differential value FA′. In this case, the surgical planning unit 164 may calculate the equipped posture with the maximum surgical planning score according to the re-descent method, based on the differential value FA′. The surgical planning unit 164 may adjust the planned position of the robot main body 320 by adopting the calculated equipped posture to optimize the equipped posture. The equipped posture with the maximum surgical planning score may be applied, for example, may be the position where the surgical planning score is equal to or greater than a threshold value th12, and the surgical planning score may be improved (become higher).

For example, when calculating the equipped posture, the surgical planning unit 164 may calculate the surgical planning score based on the port position score and determine the optimal equipped posture.

Although various embodiments have been described above with reference to the drawings, it is needless to say that the present disclosure is not limited to such examples. It is clear that a person skilled in the art can come up with various changes or modifications within the scope of the claims, and it is understood that these changes or modifications naturally belong to the technical scope of the present disclosure.

In the above-described embodiment, an example is illustrated in which the processing unit 160 of the robotically-assisted surgical device 100 performs the processing related to the preoperative simulation (for example, generation of the surgical plan), and the processing unit 360 of the robot main body 320 performs the processing related to the intraoperative navigation (for example, port registration). Instead, both processing in the preoperative simulation and the intraoperative navigation may be performed by the processing unit 160 of the robotically-assisted surgical device 100, or may be performed by the processing unit 360 of the robot main body 320.

Each threshold value may be a fixed value or a variable value. Each threshold value may be a predetermined value or a value input via the operation unit (for example, the UI 120 and the control panel CP).

The robotically-assisted surgical device 100 may include at least the processor 140 and the memory 150. The transmission/reception unit 110, the UI 120, and the display 130 may be externally attached to the robotically-assisted surgical device 100.

It is exemplified that the volume data as the captured CT image is transmitted from the CT scanner 200 to the robotically-assisted surgical device 100. Instead of this, the volume data may be transmitted to and stored in a server (for example, an image data server (PACS) (not illustrated)) or the like on the network such that the volume data is temporarily stored. In this case, the transmission/reception unit 110 of the robotically-assisted surgical device 100 may acquire the volume data from a server or the like via a wired circuit or a wireless circuit when necessary, or may acquire the volume data via any storage medium (not illustrated).

It is exemplified that the volume data as the captured CT image is transmitted from the CT scanner 200 to the robotically-assisted surgical device 100 via the transmission/reception unit 110. This also includes a case where the CT scanner 200 and the robotically-assisted surgical device 100 are established by being substantially combined into one product. This also includes a case where the robotically-assisted surgical device 100 is handled as the console of the CT scanner 200. The robotically-assisted surgical device 100 may be provided in the surgical robot 300.

Although it is exemplified that the CT scanner 200 is used to capture an image and the volume data including information on the inside of the subject is generated, the image may be captured by another device to generate the volume data. Other devices include a magnetic resonance imaging (MRI) device, a positron emission tomography (PET) device, a blood vessel imaging device (angiography device), or other modality devices. The PET device may be used in combination with other modality devices.

In the above-described embodiment, a program that realizes the function of the robotically-assisted surgical device is also applicable to a program which is supplied to the robotically-assisted surgical device via a network or various storage media, and which is read and executed by a computer in the robotically-assisted surgical device. The program that realizes the function of the surgical robot is also applicable to a program which is supplied to the surgical robot via a network or various storage media, and which is read and executed by a computer in the surgical robot.

As described above, the robotically-assisted surgical system 1 of the above-described embodiment assists robotic surgery by the surgical robot 300 having the robot main body 320, and includes the processing units 160 and 360. The processing unit 160 may plan the position of the port PT to be perforated on the body surface of the subject PS, which is the target of the robotic surgery. The processing unit 160 may acquire the captured image obtained by capturing the subject including at least a part of the subject PS by the overview camera CA included in the robot main body 320. The processing unit 160 may recognize the planned position of the port in the captured image based on the captured image and the planned position of the port PT. The processing unit 160 may display the captured image and the port position information, which indicates the planned position of the port PT in the subject PS represented in the captured image, on the display unit (for example, the control panel CP and the display device 400).

Accordingly, the robotically-assisted surgical system 1 acquires the overview image captured by the overview camera CA. After the robot main body 320 enters the operating room, at least a part of the subject PS may be reflected in the overview image. Accordingly, the robotically-assisted surgical system 1 can visualize the planned position of the port PT with respect to the subject PS using the overview image. Therefore, even when the position of the subject PS is moved for surgery, the assistant or others can perforate the port PT by confirming the display of the planned position of the port PT. In this manner, the robotically-assisted surgical system 1 can assist in perforation of the port PT, which is performed after the robot main body 320 enters the operating room. Since the camera of the overview camera CA is close to eyes of the operator in non-robotic endoscopic surgery, the overview image is in line with the intuition of the operator, and contributes to the planned surgery.

The processing unit 360 may also acquire information on the landmarks of the subject PS and the positional relationship information indicating the positional relationship between the landmarks of the subject PS and the planned position of the port PT. The processing unit 360 may recognize the image position of the landmark in the overview image. Based on the image position of the landmarks and the above-described positional relationship information, the planned position of the port PT in the overview image may be recognized.

Accordingly, the robotically-assisted surgical system 1 can easily recognize the planned position of the port PT in the overview image based on the positional relationship information between the landmark and the port PT, by using visually distinctive landmarks in the subject PS. Therefore, the robotically-assisted surgical system 1 can easily visualize the planned position of the port PT.

The processing unit 360 may also recognize the position of the perforating instrument 80 for perforating the port PT in the overview image. The processing unit 360 may show the guidance information for guiding the perforating instrument 80 to the planned position of the port PT based on the position of the perforating instrument 80 and the planned position of the port PT. Accordingly, the assistant or others involved in the surgery can easily confirm how to move the perforating instrument 80 toward the planned position of the port PT. Therefore, the robotically-assisted surgical system 1 can improve the safety when perforating the port PT.

The processing unit 160 may also acquire the 3D data of the subject PS and plan the position of the port based on the 3D data of the subject PS. The 3D data may be the volume data or model of the virtual pneumoperitoneum state, or the volume data or model of the non-pneumoperitoneum state of the subject PS. Accordingly, the robotically-assisted surgical system 1 determines the planned position of the port, taking into account the internal condition of the subject PS.

The processing unit 160 may also plan the position of the robot main body 320 with respect to the subject PS. The processing unit 360 may show the captured image obtained by capturing the subject PS in a state where the robot main body 320 is placed at the planned position, and the port position information. Accordingly, the robotically-assisted surgical system 1 visualizes the planned position of the port PT after the robot main body 320 is placed at the planned position in the operating room. Therefore, the finalized planned position of the port PT can be visualized after fixing the placement position of the robot main body 320.

The processing unit 160 may also acquire the 3D data of the subject PS and plan the position of the robot main body 320 with respect to the subject PS based on the 3D data of the subject. Accordingly, the robotically-assisted surgical system 1 can plan the optimal position of the robot main body 320 for each subject PS, taking into account the internal condition of the subject PS indicated by the 3D data.

The processing unit 360 may actuate the robot main body 320 such that the robot main body 320 is in the port perforating posture w % ben the port PT is perforated at the planned position of the port PT of the subject PS. The port perforating posture may be a posture that makes (for example, maximizes) the size of the space between the robot arm AR included in the robot main body 320 and the subject PS to be equal to or greater than the threshold value th3 (an example of a first threshold value). Accordingly, the robotically-assisted surgical system 1 can assist in perforating the port PT at the planned position of the port PT in a state where the perforating workspace is sufficiently ensured.

The processing unit 360 may actuate the robot main body 320 such that the robot main body 320 is in the equipped posture when the surgical instrument 30 included in the robot main body 320 is inserted into the subject PS through the port PT perforated on the subject PS. The equipped posture may be a posture that makes (for example, maximizes) the movable range of the surgical instruments 30 in the subject PS to be equal to or greater than the threshold value th4 (an example of a second threshold value), or a posture that makes the arm interference score (an example of the degree of interference between the robot arms AR) to be equal to or greater than the threshold value th43 (an example of a third threshold value). Accordingly, the robotically-assisted surgical system 1 can increase the degree of freedom for the operation of the surgical instrument 30 during surgery as much as possible, and to ensure as wide a working area as possible when performing various treatments using the surgical instruments 30.

The processing unit 360 may also acquire the 3D data of the subject PS and plan the equipped posture based on the 3D data of the subject PS. Accordingly, the robotically-assisted surgical system 1 can plan the optimal equipped posture for each subject PS, taking into account the internal condition of the subject PS indicated by the 3D data.

The robotically-assisted surgical system 1 may include the processing unit 160 (an example of a first processing unit) included in the robotically-assisted surgical device 100 that performs processing related to the assistance of the robotic surgery before the robotic surgery, and a processing unit (an example of a second processing unit) included in the surgical robot 300 that performs processing related to the assistance of the robotic surgery during the robotic surgery. The processing unit 160 may plan the position of the port PT. The processing unit 360 may recognize the planned position of the port PT and show the captured image and the port position information.

Accordingly, the robotically-assisted surgical system 1 can easily generate plans and the like outside the operating room, for example, before surgery, easily recognize the planned position of the port PT in the operating room during surgery, show the captured image and the port position information, and assist in perforating the port PT.

The present disclosure is advantageous for a robotically-assisted surgical system, a robotically-assisted surgical method, and a non-transitory computer-readable medium that can assist in perforation of a port, which is performed after a surgical robot enters an operating room.

1. A robotically-assisted surgical system that assists robotic surgery by a surgical robot having a robot main body, comprising:
one or more processors, wherein
the one or more processors are configured to
plan a position of a port to be perforated on a body surface of a subject which is a target of the robotic surgery,
acquire a captured image obtained by capturing the subject including at least a part of the subject by an overview camera included in the robot main body,
recognize a planned position of the port in the captured image based on the captured image and the planned position of the port, and
show the captured image and port position information indicating the planned position of the port in the subject illustrated in the captured image, on a display unit.

2. The robotically-assisted surgical system according to claim 1, wherein the one or more processors are configured to
acquire information on a landmark of the subject and positional relationship information indicating a positional relationship between the landmark of the subject and the planned position of the port,
recognize an image position of the landmark in the captured image, and
recognize the planned position of the port in the captured image based on the image position of the landmark and the positional relationship information.

3. The robotically-assisted surgical system according to claim 1, wherein the one or more processors are configured to
recognize a position of a perforating instrument for perforating the port in the captured image, and
show guidance information for guiding the perforating instrument to the planned position of the port based on the position of the perforating instrument and the planned position of the port.

4. The robotically-assisted surgical system according to claim 1, wherein the one or more processors are configured to
acquire 3D data of the subject, and
plan the position of the port based on the 3D data of the subject.

5. The robotically-assisted surgical system according to claim 1, wherein the one or more processors are configured to
plan the position of the robot main body with respect to the subject, and
show the captured image obtained by capturing the subject and the port position information in a state where the robot main body is placed at the planned position.

6. The robotically-assisted surgical system according to claim 1, wherein
the one or more processors are configured to actuate the robot main body such that the robot main body is in a port perforating posture when the port is perforated at a perforation position of the subject, and
the port perforating posture is a posture in which a size of a space between an arm provided in the robot main body and the subject is equal to or greater than a first threshold value.

7. The robotically-assisted surgical system according to claim 1, wherein
the one or more processors are configured to actuate the robot main body such that the robot main body is in an equipped posture when a surgical instrument provided in the robot main body is inserted into the subject through the port perforated in the subject, and
the equipped posture is a posture in which a size of a movable range of the surgical instrument in the subject is set to be equal to or greater than a second threshold value, or a posture in which a degree of interference between the arms provided in the robot main body is set to be equal to or less than a third threshold value.

8. The robotically-assisted surgical system according to claim 7, wherein the one or more processors are configured to
acquire 3D data of the subject, and
plan the equipped posture based on the 3D data of the subject.

9. The robotically-assisted surgical system according to claim 1,
wherein the one or more processors are configured to
perform a first processing related to assistance of the robotic surgery before the robotic surgery, and
perform a second processing related to the assistance of the robotic surgery during the robotic surgery,
wherein the one or more processors are configured to plan the position of the port in the first processing, and
wherein the one or more processors are configured to recognize the planned position of the port and shows the captured image and the port position information in the second processing.

10. A robotically-assisted surgical method that assists robotic surgery by a surgical robot having a robot main body, the robotically-assisted surgical method comprising:
planning a position of a port to be perforated on a body surface of a subject which is a target of the robotic surgery;
acquiring a captured image obtained by capturing the subject including at least a part of the subject by an overview camera included in the robot main body;
recognizing a planned position of the port in the captured image based on the captured image and the planned position of the port; and
showing the captured image and port position information indicating the planned position of the port in the subject illustrated in the captured image, on a display unit.

11. The robotically-assisted surgical method according to claim 10, further comprising:
acquiring information on a landmark of the subject and positional relationship information indicating a positional relationship between the landmark of the subject and the planned position of the port; and
recognizing an image position of the landmark in the captured image, wherein
the recognizing the planned position of the port is performed by recognizing the planned position of the port in the captured image based on the image position of the landmark and the positional relationship information.

12. The robotically-assisted surgical method according to claim 1, further comprising:
recognizing a position of a perforating instrument for perforating the port in the captured image; and
showing guidance information for guiding the perforating instrument to the planned position of the port based on the position of the perforating instrument and the planned position of the port.

13. A non-transitory computer-readable medium storing a program for causing a computer to execute a process, the process comprising:
planning a position of a port to be perforated on a body surface of a subject which is a target of robotic surgery by a surgical robot having a robot main body;
acquiring a captured image obtained by capturing the subject including at least a part of the subject by an overview camera included in the robot main body;
recognizing a planned position of the port in the captured image based on the captured image and the planned position of the port; and
showing the captured image and port position information indicating the planned position of the port in the subject illustrated in the captured image, on a display unit.

14. The non-transitory computer-readable medium according to claim 13, wherein the process comprises
acquiring information on a landmark of the subject and positional relationship information indicating a positional relationship between the landmark of the subject and the planned position of the port,
recognizing an image position of the landmark in the captured image, and
recognizing the planned position of the port in the captured image based on the image position of the landmark and the positional relationship information.

15. The non-transitory computer-readable medium according to claim 13, wherein the process comprises
recognizing a position of a perforating instrument for perforating the port in the captured image, and
showing guidance information for guiding the perforating instrument to the planned position of the port based on the position of the perforating instrument and the planned position of the port.

16. The non-transitory computer-readable medium according to claim 13, wherein the process comprises
acquiring 3D data of the subject, and
planning the position of the port based on the 3D data of the subject.

17. The non-transitory computer-readable medium according to claim 13, wherein the process comprises
planning the position of the robot main body with respect to the subject, and
showing the captured image obtained by capturing the subject and the port position information in a state where the robot main body is placed at the planned position.

18. The non-transitory computer-readable medium according to claim 13, wherein
the process comprises actuating the robot main body such that the robot main body is in a port perforating posture when the port is perforated at a perforation position of the subject, and
the port perforating posture is a posture in which a size of a space between an arm provided in the robot main body and the subject is equal to or greater than a first threshold value.

19. The non-transitory computer-readable medium according to claim 13, wherein
the process comprises actuating the robot main body such that the robot main body is in an equipped posture when a surgical instrument provided in the robot main body is inserted into the subject through the port perforated in the subject, and
the equipped posture is a posture in which a size of a movable range of the surgical instrument in the subject is set to be equal to or greater than a second threshold value, or a posture in which a degree of interference between the arms provided in the robot main body is set to be equal to or less than a third threshold value.

20. The non-transitory computer-readable medium according to claim 19, wherein the process comprises
acquiring 3D data of the subject, and
planning the equipped posture based on the 3D data of the subject.
